SEQUENCE LISTING (8 sequences)

SEQ ID NO: 1 - protein, 335 aa, Homo sapiens
  MISC_FEATURE (1)..(335): Human SLAMF1 (precursor, with signal peptide, amino acids 1-20) (UniProtKB Ref. Q13291.1, 01-OCT-2014)
MDPKGLLSLTFVLFLSLAFGASYGTGGRMMNCPKILRQLGSKVLLPLTYERINKSMNKSIHIVVTMAKSLENSVENKIVSLDPSEAGPPRYLGDRYKFYLENLTLGIRESRKEDEGWYLMTLEKNVSVQRFCLQLRLYEQVSTPEIKVLNKTQENGTCTLILGCTVEKGDHVAYSWSEKAGTHPLNPANSSHLLSLTLGPQHADNIYICTVSNPISNNSQTFSPWPGCRTDPSETKPWAVYAGLLGGVIMILIMVVILQLRRRGKTNHYQTTVEKKSLTIYAQVQKPGPLQKKLDSFPAQDPCTTIYVAATEPVPESVQETNSITVYASVTLPES

SEQ ID NO: 2 - protein, 315 aa, Homo sapiens
  MISC_FEATURE (1)..(315): Human SLAMF1 (mature, without signal peptide)
ASYGTGGRMMNCPKILRQLGSKVLLPLTYERINKSMNKSIHIVVTMAKSLENSVENKIVSLDPSEAGPPRYLGDRYKFYLENLTLGIRESRKEDEGWYLMTLEKNVSVQRFCLQLRLYEQVSTPEIKVLNKTQENGTCTLILGCTVEKGDHVAYSWSEKAGTHPLNPANSSHLLSLTLGPQHADNIYICTVSNPISNNSQTFSPWPGCRTDPSETKPWAVYAGLLGGVIMILIMVVILQLRRRGKTNHYQTTVEKKSLTIYAQVQKPGPLQKKLDSFPAQDPCTTIYVAATEPVPESVQETNSITVYASVTLPES

SEQ ID NO: 3 - protein, 237 aa, Homo sapiens
  MISC_FEATURE (1)..(237): Human SLAMF1 extracellular domain (ECD) (with signal peptide)
MDPKGLLSLTFVLFLSLAFGASYGTGGRMMNCPKILRQLGSKVLLPLTYERINKSMNKSIHIVVTMAKSLENSVENKIVSLDPSEAGPPRYLGDRYKFYLENLTLGIRESRKEDEGWYLMTLEKNVSVQRFCLQLRLYEQVSTPEIKVLNKTQENGTCTLILGCTVEKGDHVAYSWSEKAGTHPLNPANSSHLLSLTLGPQHADNIYICTVSNPISNNSQTFSPWPGCRTDPSETKP

SEQ ID NO: 4 - protein, 217 aa, Artificial Sequence
  Human SLAMF1 extracellular domain (ECD) (without signal peptide)
ASYGTGGRMMNCPKILRQLGSKVLLPLTYERINKSMNKSIHIVVTMAKSLENSVENKIVSLDPSEAGPPRYLGDRYKFYLENLTLGIRESRKEDEGWYLMTLEKNVSVQRFCLQLRLYEQVSTPEIKVLNKTQENGTCTLILGCTVEKGDHVAYSWSEKAGTHPLNPANSSHLLSLTLGPQHADNIYICTVSNPISNNSQTFSPWPGCRTDPSETKP

SEQ ID NO: 5 - protein, 451 aa, Artificial Sequence
  Human SLAMF1 ECD-Fc, without signal peptide
ASYGTGGRMMNCPKILRQLGSKVLLPLTYERINKSMNKSIHIVVTMAKSLENSVENKIVSLDPSEAGPPRYLGDRYKFYLENLTLGIRESRKEDEGWYLMTLEKNVSVQRFCLQLRLYEQVSTPEIKVLNKTQENGTCTLILGCTVEKGDHVAYSWSEKAGTHPLNPANSSHLLSLTLGPQHADNIYICTVSNPISNNSQTFSPWPGCRTDPSETKPGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 232 aa, Artificial Sequence
  Synthetic peptide Fc C237S
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 228 aa, Artificial Sequence
  Synthetic peptide Exemplary Fc #1
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 8 - protein, 229 aa, Artificial Sequence
  Synthetic peptide Exemplary Fc #2
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

(19) United States
(12) Patent Application Publication
(10) Pub. No.: US 2018/0016555 A1
(43) Pub. Date: Jan. 18, 2018

(54) SLAMF1 ANTAGONISTS AND USES THEREOF

(71) Applicant: Five Prime Therapeutics, Inc., South San Francisco, CA (US)

(72) Inventors: Luis Borges, San Mateo, CA (US); Nathan Sallee, San Francisco, CA (US); Charles Kaplan, San Francisco, CA (US); Arthur Brace, Redwood City, CA (US); W. Michael Kavanaugh, Orinda, CA (US); Brian Wong, Los Altos, CA (US); David Bellovin, Sunnyvale, CA (US); Thomas Brennan, San Jose, CA (US); Artur Karasyov, Oakland, CA (US)

(73) Assignee: Five Prime Therapeutics, Inc., South San Francisco, CA (US)

(21) Appl. No.: 15/520,998

(22) PCT Filed: Oct. 21, 2015
(86) PCT No.: PCT/US2015/056714

§ 371 Date: Apr. 21, 2017

Related U.S. Application Data
(60) Provisional application No. 62/067,638, filed on Oct. 23, 2014.
(60) Provisional application No. 62/155,810, filed on May 1, 2015.

(51) Int. Cl. C12N 5/0783; C07K 16/28; G01N 33/50

(52) U.S. Cl. CPC C12N 5/0636; G01N 33/505; C07K 16/2896; C07K 2317/76; C12N 2501/599

(57) ABSTRACT

Methods of identifying and using SLAMF1 antagonists are provided.

### CROSS-REFERENCE TO RELATED APPLICATIONS

This application claims the benefit of priority of US Provisional Application No. 62/067,638, filed Oct. 23, 2014, and US Provisional Application No. 62/155,810, filed May 1, 2015, each of which is incorporated by reference herein in its entirety for any purpose.

### TECHNICAL FIELD

Methods of identifying and using SLAMF1 antagonists are provided. Such methods include, but are not limited to, methods of treating cancer. SLAMF1 antagonists include, but are not limited to, antibodies that bind SLAMF1.

### BACKGROUND

Genetic alterations in cancer provide a diverse set of antigens that can mediate anti-tumor immunity. Antigen recognition through T-cell receptors (TCRs) initiate T-cell-responses, which are regulated by a balance between activating and inhibitory signals. The inhibitory signals, or “immune checkpoints,” play an important role in normal tissues by preventing autoimmunity. Up-regulation of immune checkpoint proteins allows cancers to evade anti-tumor immunity. Two immune checkpoint proteins have been the focus of clinical cancer immunotherapeutics, cytotoxic T-lymphocyte-associated antigen 4 (CTLA4) and programmed cell death protein 1 (PD1). An anti-CTLA4 antibody has been approved for treatment of metastatic melanoma and is currently in clinical trials for other cancers. Anti-PD-1 antibody and anti-PD-L1 antibody, which is directed to the ligand for PD-1, are also currently in clinical development.

Identification of further proteins involved in T cell activation and inhibition would assist in further understanding T-cell responses, and provide many advantages to drug development including selection of therapeutically effective and safe therapeutics, biomarkers for patient selection and companion diagnostics, targets for combination therapy, and new targets for developing cancer immunotherapeutic agents.

### SUMMARY

In some embodiments, methods of treating cancer are provided. In some embodiments, a method of treating cancer comprises administering to a subject with cancer an effective amount of at least one SLAMF1 antagonist. In some embodiments, methods of inhibiting suppression of activated T cells are provided. In some embodiments, a method comprises administering to a subject at least one SLAMF1 antagonist.

In some embodiments, methods of treating cancer and/or inhibiting suppression of activated T cells further comprises administering to the subject an effective amount of a therapeutic agent selected from chemotherapeutic agents, anti-angiogenesis agents, growth inhibitory agents, and anti-neoplastic compositions. In some embodiments, the anti-neoplastic composition comprises an immune stimulating agent. In some embodiments, the immune stimulating agent is chosen from agents falling within one or more of the following categories:

a) an agonist of an immune stimulatory molecule, including a co-stimulatory molecule, such as an immune-stimulatory molecule found on a T cell or NK cell;

b) an antagonist of an immune inhibitory molecule, including a co-inhibitory molecule, such as an immune-stimulatory molecule found on a T cell or NK cell;

c) an antagonist of CTLA4, LAG-3, PD-1, PDL1, PDL2, Galectin 1, Galectin 9, CEACAM-1, BTLA, CD25, CD69, TIGIT, CD113, GPR56, VISTA, B7-H3, B7-H4, 2B4, CD48, GARP, PD1H, LAIR1, TIM1, TIM3, TIM4, ILT4, IL-6, IL-10, TGFβ, VEGF, KIR, adenosine A2A receptor, PI3Kdelta, or IDO;

d) an agonist of B7-1, B7-2, CD28, 4-1BB (CD137), 4-1BBL, ICOS, ICOS-L, OX40, OX40L, GITR, GITRL, CD27, CD40, CD40L, DR3, CD28H, IL-2, IL-7, IL-12, IL-15, IL-21, IFNα, STING, or a Toll-like receptor agonist such as a TLR2/4 agonist; e) an agent that binds to a member of the B7 family of membrane-bound proteins such as B7-1, B7-2, B7-H2 (ICOS-L), B7-H3, B7-H4, B7-H5 (VISTA), and B7-H6;

f) an agent that binds to a member of the TNF receptor family or a co-stimulatory or co-inhibitory molecule binding to a member of the TNF receptor family such as CD40, CD4OL, OX40, OX4OL, GITR, GITRL, CD70, CD27L, CD30, CD3OL, 4-1BBL, CD137 (4-1BB), TRAIL/Apo2-L, TRAILR1/DR4, TRAILR2/DR5, TRAILR3, TRAILR4, OPG, RANK, RANKL, TWEAKR/Fn14, TWEAK, BAFFR, EDAR, XEDAR, EDA1, EDA2, TACI, APRIL, BCMA, LTβR, LIGHT, DeR3, HVEM, VEGL/TL1A, TRAMP/DR3, TNFR1, TNFβ, TNFR2, TNFα, 1β2, FAS, FASL, RELT, DR6, TROY, or NGFβ;

g) an agent that antagonizes or inhibits a cytokine that inhibits T cell activation such as IL-6, IL-10, TGFβ, VEGF;

h) an agonist of a cytokine that stimulates T cell activation such as IL-2, IL-7, IL-12, IL-15, IL-21, and IFNα; and

i) an antagonist of a chemokine, such as CXCR2, CXCR4, CCR2, or CCR4.

In some embodiments, methods of inhibiting suppression of activated T cells are provided, comprising contacting the T cells with at least one SLAMF1 antagonist. In some such embodiments, the T cells are in vitro.

In some embodiments, a SLAMF1 antagonist reduces suppression of proliferation of activated T cells by at least 30%, at least 40%, at least 50%, at least 60%, at least 70%, or at least 80%. In some embodiments, the activated T cells are CD3+T cells. In some embodiments, the activated T cells are IL-2-activated CD3+T cells.

In any of the embodiments described herein, the SLAMF1 antagonist may be a SLAMF1 extracellular domain (ECD) or a SLAMF1 ECD fusion molecule. In some embodiments, the SLAMF1 ECD or SLAMF1 ECD fusion molecule is monomeric. In some embodiments, the SLAMF1 ECD or SLAMF1 ECD fusion molecule is dimeric.

In any of the embodiments described herein, the SLAMF1 antagonist may be a SLAMF1 antibody. In some embodiments, the antibody is selected from a chimeric antibody, a humanized antibody, and a human antibody. In some embodiments, the antibody is a bispecific antibody or a single chain antibody. In some embodiments, the antibody is an antibody fragment. In some embodiments, the antibody fragment is selected from an Fv, a single-chain Fv (scFv), a Fab, a Fab′, and a (Fab′)₂.

In any of the embodiments described herein, the SLAMF1 antagonist may be a small molecule or a small peptide.

In some embodiments, methods of identifying a SLAMF1 antagonist are provided. In some embodiments, the method comprises:

a) contacting activated T cells with a candidate molecule and a SLAMF1 molecule, wherein the SLAMF1 molecule comprises SLAMF1, a SLAMF1 ECD, or a SLAMF1 ECD fusion molecule; and

b) detecting proliferation of the activated T cells;

wherein a reduction in suppression of proliferation of the activated T cells in the presence of the candidate molecule as compared to suppression of proliferation of the activated T cells in the absence of the candidate molecule indicates that the candidate molecule is a SLAMF1 antagonist. In some embodiments, suppression of proliferation of activated T cells is reduced by at least 30%, at least 40%, at least 50%, at least 60%, at least 70%, or at least 80% in the presence of the candidate molecule. In some embodiments, the candidate molecule binds to SLAMF1. In some embodiments, the candidate molecule is an antibody that binds SLAMF1. In some embodiments, the candidate molecule is a small molecule. In some embodiments, the candidate molecule is a small peptide. In some embodiments, the activated T cells are activated CD3+T cells. In some embodiments, the activated T cells are IL-2-activated CD3+T cells.

In some embodiments, methods of determining whether a SLAMF1 antibody is a SLAMF1 antagonist are provided. In some embodiments, the method comrpises:

a) contacting activated T cells with the SLAMF1 antibody and a SLAMF1 molecule, wherein the SLAMF1 molecule comprises SLAMF1, a SLAMF1 ECD, or a SLAMF1 ECD fusion molecule; and

b) detecting proliferation of the activated T cells;

wherein a reduction in suppression of proliferation of the activated T cells in the presence of the SLAMF1 antibody as compared to suppression of proliferation of the activated T cells in the absence of the SLAMF1 antibody indicates that the SLAMF1 antibody is a SLAMF1 antagonist. In some embodiments, suppression of proliferation of activated T cells is reduced by at least 30%, at least 40%, at least 50%, at least 60%, at least 70%, or at least 80% in the presence of the SLAMF1 antibody. In some embodiments, the activated T cells are activated CD3+T cells. In some embodiments, the activated T cells are IL-2-activated CD3+ T cells.

In some embodiments, use of a SLAMF1 antagonist for treating cancer in a subject is provided. In some such embodiments, the SLAMF1 antagonist is a SLAMF1 antibody. In some embodiments, the antibody is selected from a chimeric antibody, a humanized antibody, and a human antibody. In some embodiments, the antibody is an antibody fragment. In some embodiments, the antibody fragment is selected from an Fv, a single-chain Fv (scFv), a Fab, a Fab′, and a (Fab′)2. In some embodiments, the antibody is a bispecific antibody or a single chain antibody. In some embodiments, the SLAMF1 antagonist is a SLAMF1 extracellular domain (ECD) or a SLAMF1 ECD fusion molecule. In some embodiments, the SLAMF1 ECD or SLAMF1 ECD fusion molecule is monomeric. In some embodiments, the SLAMF1 ECD or SLAMF1 ECD fusion molecule is dimeric. In some embodiments, the SLAMF1 antagonist is a small molecule or a small peptide.

Any embodiment described herein or any combination thereof applies to any and all methods of the invention described herein.

### BRIEF DESCRIPTION OF THE FIGURES

FIG. 1. PD-L1 suppresses proliferation of activated/IL-2 rested T cells. CD3+ T cells were activated for 6 days with atni-CD3/anti-CD28 beads and rested for an additional 4 days in IL-2. Following resting, T cells were re-stimulated on plates that had been coated with anti-CD3, anti-human IgG and titrating doses of Fc-protein (starting at 100μg/mL; 1:3 dilutions) for 3 days at 37° C. Cells were pulsed with Edu 12-16 hours prior to harvesting cells and changes in proliferation were quantified by the number of cells that had incorporated Edu as measured by FACS. Each panel shows the results using CD3+ T cells from a different donor.

FIG. 2. SLAMF1-Fc suppresses proliferation of activated/IL-2 rested T cells. CD3 T cells were activated for 6 days with atni-CD3/anti-CD28 beads and rested for an additional 4 days in IL-2. Following resting, T cells were re-stimulated on plates that had been coated with anti-CD3, anti-human IgG and titrating doses of Fc-protein (starting at 100μg/mL; 1:3 dilutions) for 3d at 37° C. Cells were pulsed with Edu 12-16 hours prior to harvesting cells and changes in proliferation were quantified by the number of cells that had incorporated Edu as measured by FACS. Each panel shows the results using CD3+T cells from a different donor.

FIG. 3. Expression of SLAMF1 mRNA in human tissues.

FIG. 4. SLAMF1 ECD Alters the Growth of E.G7-OVA Tumors In Vivo. A. C57BL/6 mice were induced to express constitutive, systemic expression of SLAMF1 ECD. Mice expressing the ECD or treated with saline as a control were inoculated with E.G7-OVA murine T-cell lymphoma cells, and tumor volume was measured. Tumors grown in mice expressing SLAMF1 ECD were significantly larger than those in saline-treated mice on Days 15 and 18 (* p<0.05). B. Individual E.G7-OVA tumor volumes as assessed on Day 18 are shown. Statistical significance was determined via two-tailed, unpaired t-Test.

FIG. 5A-C. Anti-SLAMF1 blocking antibodies relieve T cell inhibition in an A20 APC assay. A. A schematic diagram of the A20 artificial APC assay is shown. B. Transduction of SLAMF1 into A20 cells reduces T cell proliferation stimulated by OCA peptide. C. Inhibition of T cell proliferation by SLAMF1 is reversed by blocking anti-SLAMF1 antibodies.

FIG. 6A-D. Anti-SLAMF1 blocking antibodies stimulate release of interferon gamma (INFγ) from human CD8+cells co-incubated with T2 cells. A. Polyclonal anti-SLAMF1 antibodies stimulate dose-dependent release of interferon-gamma. B. Monoclonal anti-SLAMF1 antibodies also stimulate release of interferon. C. Pre-incubation of T2 cells, but not CD8 cells, with polyclonal anti-SLAMF1 antibody induces IFNγ release. D. Anti-SLAMF1 blocking antibody stimulation of INFγ release is not blocked by anti-CD32 antibody.

FIG. 7A-C. SLAMF1 is expressed on tumor-infiltrating T cells. A. SLAMF1 is expressed on CD4+and a subset of CD8+T cells. B. SLAMF1 is expressed on T regulatory cells. C. PD-1 and SLAMF1 are both expressed on the majority of CD4 T cells.

### DEIAILED DESCRIPTION

The present inventors have identified SLAMF1 as a suppressor of activated T cells. SLAMF1 is expressed on activated CD4+and CD8+T cells and it belongs to a family of 9 proteins that have two extracellular immunoglobulin domains and the majority of which have intracellular immunoreceptor tyrosine-based switch motifs (ITSMs). Without intending to be bound by any particular theory, SLAMF1 may interact with T cells, resulting in an inhibitory signal in the T cells that induces an inactive state (e.g., an anergic or tolerized state). Inhibition of SLAMF1 activity, for example, with an inhibitory antibody or soluble SLAMF1, may therefore enhance immune-mediated killing of cancer cells. Targeting molecules include antibodies that bind SLAMF1 and block SLAMF1 activity and SLAMF1 extracellular domain (ECD) and SLAMF1 ECD fusion proteins, including monomeric SLAMF1 ECD and SLAMF1 ECD fusion proteins. Such targeting molecules are provided as therapeutic agents for treating cancer.

All references cited herein, including patent applications and publications, are incorporated by reference herein in their entirety.

The section headings used herein are for organizational purposes only and are not to be construed as limiting the subject matter described.

### Definitions

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular.

Exemplary techniques used in connection with recombinant DNA, oligonucleotide synthesis, tissue culture and transformation (e.g., electroporation, lipofection), enzymatic reactions, and purification techniques are known in the art. Many such techniques and procedures are described, e.g., in Sambrook et al. Molecular Cloning: A Laboratory Manual (3rd ed., Cold Spring Harbor Laboratory Press, Cold Spring Harbor, N.Y. (2001)), among other places. In addition, exemplary techniques for chemical syntheses, chemical analyses, pharmaceutical preparation, formulation, and delivery, and treatment of patients are also known in the art.

In this application, the use of “or” means “and/or” unless stated otherwise. In the context of a multiple dependent claim, the use of “or” refers back to more than one preceding independent or dependent claim in the alternative only. Unless otherwise indicated, the term “include” has the same meaning as “include, but are not limited to,” the term “includes” has the same meaning as “includes, but is not limited to,” and the term “including” has the same meaning as “including, but not limited to.” Similarly, the term “such as” has the same meaning as the term “such as, but not limited to.” Also, terms such as “element” or “component” encompass both elements and components comprising one unit and elements and components that comprise more than one subunit unless specifically stated otherwise.

As utilized in accordance with the present disclosure, the following terms, unless otherwise indicated, shall be understood to have the following meanings:

The terms “nucleic acid molecule” and “polynucleotide” may be used interchangeably, and refer to a polymer of nucleotides. Such polymers of nucleotides may contain natural and/or non-natural nucleotides, and include, but are not limited to, DNA, RNA, and PNA. “Nucleic acid sequence” refers to the linear sequence of nucleotides that comprise the nucleic acid molecule or polynucleotide.

The terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present invention, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification. A “small peptide” refers to a peptide having 50 or fewer amino acids. In some embodiments, a small peptide has 40 or fewer, or 35 or fewer, or 30 or fewer, or 25 or fewer amino acids. In some embodiments, a small peptide has 10 to 50 amino acids or 15 to 30 amino acids.

A “native sequence” polypeptide comprises a polypeptide having the same amino acid sequence as a polypeptide found in nature. Thus, a native sequence polypeptide can have the amino acid sequence of naturally occurring polypeptide from any mammal. Such native sequence polypeptide can be isolated from nature or can be produced by recombinant or synthetic means. The term “native sequence” polypeptide specifically encompasses naturally occurring truncated or secreted forms of the polypeptide (e.g., an extracellular domain sequence), naturally occurring variant forms (e.g., alternatively spliced forms) and naturally occurring allelic variants of the polypeptide.

A polypeptide “variant” means a biologically active polypeptide having at least about 80% amino acid sequence identity with the native sequence polypeptide after aligning the sequences and introducing gaps, if necessary, to achieve the maximum percent sequence identity, and not considering any conservative substitutions as part of the sequence identity. Such variants include, for instance, polypeptides wherein one or more amino acid residues are added, or deleted, at the N- or C-terminus of the polypeptide. In some embodiments, a variant will have at least about 80% amino acid sequence identity. In some embodiment, a variant will have at least about 90% amino acid sequence identity. In some embodiment, a variant will have at least about 95% amino acid sequence identity with the native sequence polypeptide. In some embodiment, a variant will have at least about 97% amino acid sequence identity with the native sequence polypeptide.

As used herein, “Percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGNTM (DNASTAR) software. Those skilled in the art can determine appropriate parameters for measuring alignment, including any algorithms needed to achieve maximal alignment over the full length of the sequences being compared.

The terms “signaling lymphocytic activation molecule” and “SLAMF1” are used interchangeably and include any native SLAMF1 from any vertebrate source, including mammals such as primates (e.g. humans) and rodents (e.g., mice and rats), unless otherwise indicated. The term includes full-length, unprocessed SLAMF1 as well as any form of SLAMF1 that results from processing in the cell or any fragment thereof that retains activity (e.g., suppression of activated CD3+T cells). The term also encompasses naturally occurring variants of SLAMF1, e.g., splice variants or allelic variants. In some embodiments, SLAMF1 is a human SLAMF1 with an amino acid sequence of SEQ ID NO: 1 (precursor, with signal peptide) or an amino acid sequence of SEQ ID NO: 2 (mature, without signal peptide).

The term “SLAMF1” also includes full-length SLAMF1, SLAMF1 fragments, and SLAMF1 variants. The term “full-length SLAMF1”, as used herein, refers to full-length, unprocessed SLAMF1 as well as any form of SLAMF1 that results from processing in the cell or any fragment thereof that retains activity (e.g., suppression of activated CD3+T cells). In some embodiments, a full-length human SLAMF1 has the amino acid sequence of SEQ ID NO: 1 (precursor, with signal peptide) or SEQ ID NO: 2 (mature, without signal peptide). As used herein, the term “SLAMF1 fragment” refers to SLAMF1 having one or more residues deleted from the N- and/or C-terminus of the full-length SLAMF1 and that retains activity. As used herein, the term “SLAMF1 variant” refers to SLAMF1 that contains amino acid additions, deletions, and substitutions and that remain active. Such variants may be at least 80%, 85%, 90%, 92%, 95%, 97%, 98%, or 99% identical to the parent SLAMF1. The % identity of two polypeptides can be measured by a similarity score determined by comparing the amino acid sequences of the two polypeptides using the Bestfit program with the default settings for determining similarity. Bestfit uses the local homology algorithm of Smith and Waterman, Advances in Applied Mathematics 2:482-489 (1981) to find the best segment of similarity between two sequences.

The term “antagonist” is used in the broadest sense, and includes any molecule that partially or fully inhibits or neutralizes a biological activity of a polypeptide, such as SLAMF1, or that partially or fully inhibits the transcription or translation of a nucleic acid encoding the polypeptide. Exemplary antagonist molecules include, but are not limited to, antagonist antibodies, SLAMF1 extracellular domain (ECD) proteins and fusion molecules, small peptides, oligopeptides, organic molecules (including small molecules), aptamers, and antisense nucleic acids. In some embodiments, an antagonist agent may be referred to as a blocking agent (such as a blocking antibody).

The term “SLAMF1 antagonist” refers to a molecule that interacts with SLAMF1 and inhibits SLAMF1-mediated signaling or activity (such activity including, but not limited to, suppression of activated CD3+T cells). Exemplary SLAMF1 antagonists include antibodies that bind SLAMF1, soluble SLAMF1 extracellular domain (ECD) protein, and SLAMF1 ECD fusion molecules. In some embodiments, SLAMF1 ECD and SLAMF1 ECD fusion molecules are monomeric. In some embodiments, SLAMF1 ECD and SLAMF1 ECD fusion molecules are dimeric.

A SLAMF1 antagonist is considered to “inhibit SLAMF1 activity” when it reduces SLAMF1-mediated suppression of activated T cells by at least 50%. In some embodiments, a SLAMF1 antagonist reduces SLAMF1-mediated suppression of activated T cells by at least 50% using the assay described in Example 3. In some embodiments, a SLAMF1 antagonist reduces SLAMF1-mediated suppression of activated T cells by at least 60%, at least 70%, at least 80%, or at least 90%.

The terms “inhibition” or “inhibit” refer to a decrease or cessation of any phenotypic characteristic or to the decrease or cessation in the incidence, degree, or likelihood of that characteristic. In some embodiments, by “reduce” or “inhibit” is meant the ability to cause a decrease of 20% or greater. In another embodiment, by “reduce” or “inhibit” is meant the ability to cause a decrease of 50% or greater. In yet another embodiment, by “reduce” or “inhibit” is meant the ability to cause an overall decrease of 75%, 85%, 90%, 95%, or greater.

The term “SLAMF1 antibody” or “antibody that binds SLAMF1,” as used herein, refers to an antibody that binds to SLAMF1. In some embodiments, a SLAMF1 antibody inhibits SLAMF1-mediated signaling or activity. In some embodiments, a SLAMF1 antibody refers to an antibody that is capable of binding SLAMF1 with sufficient affinity such that the antibody is useful as a diagnostic and/or therapeutic agent in targeting SLAMF1. In some embodiments, the extent of binding of a SLAMF1 antibody to an unrelated, non-SLAMF1 protein is less than about 10% of the binding of the antibody to SLAMF1 as measured, e.g., by a radioimmunoassay (RIA). In some embodiments, a SLAMF1 antibody binds to an epitope of SLAMF1 that is conserved among SLAMF1 from different species. In some embodiments, a SLAMF1 antibody binds to the same epitope as a human or humanized SLAMF1 antibody that binds human SLAMF1.

The term “antibody” herein is used in the broadest sense and encompasses various antibody structures, including but not limited to monoclonal antibodies, polyclonal antibodies, multispecific antibodies (e.g., bispecific antibodies), single-chain antibodies (e.g., camelid antibodies), fibronectin type III scaffold antibodies (such as AdnectinsTM; see, e.g., Lipovsek, 2011, Prot. Eng. Des. Sel. 24: 3-9), and antibody fragments so long as they exhibit the desired antigen-binding activity. The term “antibody” as used herein further refers to a molecule comprising complementarity-determining region (CDR) 1, CDR2, and CDR3 of a heavy chain and CDR1, CDR2, and CDR3 of a light chain, wherein the molecule is capable of binding to antigen. The term antibody includes, but is not limited to, fragments that are capable of binding antigen, such as Fv, single-chain Fv (scFv), Fab, Fab′, and (Fab′)2. The term antibody also includes, but is not limited to, chimeric antibodies, humanized antibodies, and antibodies of various species such as mouse, human, cynomolgus monkey, etc.

In some embodiments, an antibody comprises a heavy chain variable region and a light chain variable region. In some embodiments, an antibody comprises at least one heavy chain comprising a heavy chain variable region and at least a portion of a heavy chain constant region, and at least one light chain comprising a light chain variable region and at least a portion of a light chain constant region. In some embodiments, an antibody comprises two heavy chains, wherein each heavy chain comprises a heavy chain variable region and at least a portion of a heavy chain constant region, and two light chains, wherein each light chain comprises a light chain variable region and at least a portion of a light chain constant region. As used herein, a single-chain Fv (scFv), or any other antibody that comprises, for example, a single polypeptide chain comprising all six CDRs (three heavy chain CDRs and three light chain CDRs) is considered to have a heavy chain and a light chain. In some such embodiments, the heavy chain is the region of the antibody that comprises the three heavy chain CDRs and the light chain in the region of the antibody that comprises the three light chain CDRs.

The term “heavy chain variable region” as used herein refers to a region comprising heavy chain CDR1, framework (FR) 2, CDR2, FR3, and CDR3. In some embodiments, a heavy chain variable region also comprises at least a portion of an FR1, which is N-terminal to CDR1, and/or at least a portion of an FR4, which is C-terminal to CDR3.

The term “heavy chain constant region” as used herein refers to a region comprising at least three heavy chain constant domains, C_{H}1, C_{H}2, and C_{H}3. Nonlimiting exemplary heavy chain constant regions include γ, δ, and α. Nonlimiting exemplary heavy chain constant regions also include ε and μ. Each heavy constant region corresponds to an antibody isotype. For example, an antibody comprising a γ constant region is an IgG antibody, an antibody comprising a δ constant region is an IgD antibody, and an antibody comprising an a constant region is an IgA antibody. Further, an antibody comprising a μ constant region is an IgM antibody, and an antibody comprising an ε constant region is an IgE antibody. Certain isotypes can be further subdivided into subclasses. For example, IgG antibodies include, but are not limited to, IgG1 (comprising a γ₁ constant region), IgG2 (comprising a γ₂ constant region), IgG3 (comprising a γ₃ constant region), and IgG4 (comprising a γ₄ constant region) antibodies; IgA antibodies include, but are not limited to, IgA1 (comprising an α₁ constant region) and IgA2 (comprising an α₂ constant region) antibodies; and IgM antibodies include, but are not limited to, IgM1 and IgM2.

The term “heavy chain” as used herein refers to a polypeptide comprising at least a heavy chain variable region, with or without a leader sequence. In some embodiments, a heavy chain comprises at least a portion of a heavy chain constant region. The term “full-length heavy chain” as used herein refers to a polypeptide comprising a heavy chain variable region and a heavy chain constant region, with or without a leader sequence.

The term “light chain variable region” as used herein refers to a region comprising light chain CDR1, framework (FR) 2, CDR2, FR3, and CDR3. In some embodiments, a light chain variable region also comprises an FR1 and/or an FR4.

The term “light chain constant region” as used herein refers to a region comprising a light chain constant domain, C_{L}. Nonlimiting exemplary light chain constant regions include γ and κ.

The term “light chain” as used herein refers to a polypeptide comprising at least a light chain variable region, with or without a leader sequence. In some embodiments, a light chain comprises at least a portion of a light chain constant region. The term “full-length light chain” as used herein refers to a polypeptide comprising a light chain variable region and a light chain constant region, with or without a leader sequence.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. The term “compete” when used in the context of an antibody that compete for the same epitope means competition between antibodies is determined by an assay in which an antibody being tested prevents or inhibits specific binding of a reference antibody to a common antigen (e.g., SLAMF1). Numerous types of competitive binding assays can be used, for example: solid phase direct or indirect radioimmunoassay (RIA), solid phase direct or indirect enzyme immunoassay (EIA), sandwich competition assay (see, e.g., Stahli et al., 1983, Methods in Enzymology 9:242-253); solid phase direct biotin-avidin EIA (see, e.g., Kirkland et al., 1986, J. Immunol. 137:3614-3619) solid phase direct labeled assay, solid phase direct labeled sandwich assay (see, e.g., Harlow and Lane, 1988, Antibodies, A Laboratory Manual, Cold Spring Harbor Press); solid phase direct label RIA using 1-125 label (see, e.g., Morel et al., 1988, Molec. Immunol. 25:7-15); solid phase direct biotin-avidin EIA (see, e.g., Cheung, et al., 1990, Virology 176:546-552); and direct labeled RIA (Moldenhauer et al., 1990, Scand. J. Immunol. 32:77-82). Typically, such an assay involves the use of purified antigen bound to a solid surface or cells bearing either of these, an unlabeled test antigen binding protein and a labeled reference antibody. Competitive inhibition is measured by determining the amount of label bound to the solid surface or cells in the presence of the test antibody. Usually the test antibody is present in excess. Antibodies identified by competition assay (competing antibodies) include antibodies binding to the same epitope as the reference antibodies and antibodies binding to an adjacent epitope sufficiently proximal to the epitope bound by the reference antibody for steric hindrance to occur. In some embodiments, when a competing antibody is present in excess, it will inhibit specific binding of a reference antibody to a common antigen by at least 40%, 45%, 50%, 55%, 60%, 65%, 70% or 75%. In some instance, binding is inhibited by at least 80%, 85%, 90%, 95%, or 97% or more.

The term “antigen” refers to a molecule or a portion of a molecule capable of being bound by a selective binding agent, such as an antibody or immunologically functional fragment thereof, and additionally capable of being used in a mammal to produce antibodies capable of binding to that antigen. An antigen may possess one or more epitopes that are capable of interacting with antibodies.

The term “epitope” is the portion of a molecule that is bound by a selective binding agent, such as an antibody or a fragment thereof The term includes any determinant capable of specifically binding to an antibody. An epitope can be contiguous or non-contiguous (e.g., in a polypeptide, amino acid residues that are not contiguous to one another in the polypeptide sequence but that within in context of the molecule are bound by the antigen binding protein). In some embodiments, epitopes may be mimetic in that they comprise a three dimensional structure that is similar to an epitope used to generate the antibody, yet comprise none or only some of the amino acid residues found in that epitope used to generate the antibody. Epitope determinants may include chemically active surface groupings of molecules such as amino acids, sugar side chains, phosphoryl or sulfonyl groups, and may have specific three dimensional structural characteristics, and/or specific charge characteristics.

A “chimeric antibody” as used herein refers to an antibody comprising at least one variable region from a first species (such as mouse, rat, cynomolgus monkey, etc.) and at least one constant region from a second species (such as human, cynomolgus monkey, chicken, etc.). In some embodiments, a chimeric antibody comprises at least one mouse variable region and at least one human constant region. In some embodiments, a chimeric antibody comprises at least one cynomolgus variable region and at least one human constant region. In some embodiments, all of the variable regions of a chimeric antibody are from a first species and all of the constant regions of the chimeric antibody are from a second species.

A “humanized antibody” as used herein refers to an antibody in which at least one amino acid in a framework region of a non-human variable region (such as mouse, rat, cynomolgus monkey, chicken, etc.) has been replaced with the corresponding amino acid from a human variable region. In some embodiments, a humanized antibody comprises at least one human constant region or fragment thereof In some embodiments, a humanized antibody is an Fab, an scFv, a (Fab′)₂, etc.

A “CDR-grafted antibody” as used herein refers to a humanized antibody in which one or more complementarity determining regions (CDRs) of a first (non-human) species have been grafted onto the framework regions (FRs) of a second (human) species.

A “human antibody” as used herein refers to antibodies produced in humans, antibodies produced in non-human animals that comprise human immunoglobulin genes, such as XenoMouse®, and antibodies selected using in vitro methods, such as phage display, wherein the antibody repertoire is based on a human immunoglobulin sequences.

The term “SLAMF1 extracellular domain” (“SLAMF1 ECD”) includes full-length SLAMF1 ECDs, SLAMF1 ECD fragments, and SLAMF1 ECD variants, and refers to a SLAMF1 polypeptide that lacks the intracellular and transmembrane domains. In some embodiments, the SLAMF1 ECD is capable of binding SLAMF1′s binding partner on T cells. The term “full-length SLAMF1 ECD”, as used herein, refers to a SLAMF1 ECD that extends to the last amino acid of the extracellular domain, and includes natural splice variants in the extracellular domain. The full-length SLAMF1 ECD may or may not comprise a signal peptide. In some embodiments, a full-length SLAMF1 ECD has the amino acid sequence of SEQ ID NO: 3 (with signal peptide) or SEQ ID NO: 4 (without signal peptide). As used herein, the term “SLAMF1 ECD fragment” refers to a SLAMF1 ECD having one or more residues deleted from the N- and/or C-terminus of the full-length ECD. A SLAMF1 ECD fragment may or may not comprise a signal peptide. As used herein, the term “SLAMF1 ECD variants” refers to SLAMF1 ECDs that contain amino acid additions, deletions, and substitutions. Such variants may be at least 80%, 85%, 90%, 92%, 95%, 97%, 98%, or 99% identical to the parent SLAMF1 ECD. The % identity of two polypeptides can be measured by a similarity score determined by comparing the amino acid sequences of the two polypeptides using the Bestfit program with the default settings for determining similarity. Bestfit uses the local homology algorithm of Smith and Waterman, Advances in Applied Mathematics 2:482-489 (1981) to find the best segment of similarity between two sequences. In some embodiments, a SLAMF1 ECD is monomeric. In some embodiments, a SLAMF1 ECD is dimeric.

Without intending to be bound by any particular theory, it is believed that a soluble SLAMF1 ECD or SLAMF1 ECD fusion molecule would be an antagonist of SLAMF1 activity, while a substrate-bound SLAMF1 ECD or SLAMF1 ECD fusion molecule (such as an SLAMF1 ECD or SLAMF1 ECD fusion molecule bound to a solid surface) acts as an SLAMF1 mimic (see, e.g., Example 3). The distinction between a soluble SLAMF1 ECD or SLAMF1 ECD fusion molecule and a substrate-bound SLAMF1 ECD or SLAMF1 ECD fusion molecule may, in some instances, lie in the ability of the substrate-bound SLAMF1 ECD or SLAMF1 ECD fusion molecule to cross-link the inhibitory receptor on the surface of the T cells.

The term “SLAMF1 ECD fusion molecule” refers to a molecule comprising a SLAMF1 ECD, and one or more “fusion partners.” In some embodiments, the SLAMF1 ECD fusion molecule is capable of binding SLAMF1′s binding partner on T cells. In some embodiment, the SLAMF1 ECD and the fusion partner are covalently linked (“fused”). If the fusion partner is also a polypeptide (“the fusion partner polypeptide”), the SLAMF1 ECD and the fusion partner polypeptide may be part of a continuous amino acid sequence, and the fusion partner polypeptide may be linked to either the N-terminus or the C-terminus of the SLAMF1 ECD. In such cases, the SLAMF1 ECD and the fusion partner polypeptide may be translated as a single polypeptide from a coding sequence that encodes both the SLAMF1 ECD and the fusion partner polypeptide (the “SLAMF1 ECD fusion protein”). In some embodiments, the SLAMF1 ECD and the fusion partner are covalently linked through other means, such as, for example, a chemical linkage other than a peptide bond. Many known methods of covalently linking polypeptides to other molecules (for example, fusion partners) may be used. In other embodiments, the SLAMF1 ECD and the fusion partner may be fused through a “linker,” which is comprised of at least one amino acid or chemical moiety. A nonlimiting exemplary SLAMF1 ECD fusion molecule has the sequence of SEQ ID NO: 5. In some embodiments, a SLAMF1 ECD fusion molecule is monomeric. In some embodiments, a SLAMF1 ECD fusion molecule is dimeric. In some embodiments, the fusion partner is linked to the N-terminus of a SLAMF1 ECD.

In some embodiments, the SLAMF1 polypeptide and the fusion partner are noncovalently linked. In some such embodiments, they may be linked, for example, using binding pairs. Exemplary binding pairs include, but are not limited to, biotin and avidin or streptavidin, an antibody and its antigen, etc.

Exemplary fusion partners include, but are not limited to, an immunoglobulin Fc domain, albumin, and polyethylene glycol. The amino acid sequences of nonlimiting exemplary Fc domains are shown in SEQ ID NOs: 6 to 8.

In some embodiments, a SLAMF1 ECD amino acid sequence is derived from that of a non-human mammal. In such embodiments, the SLAMF1 ECD amino acid sequence may be derived from mammals including, but not limited to, rodents (including mice, rats, hamsters), rabbits, simians, felines, canines, equines, bovines, porcines, ovines, caprines, mammalian laboratory animals, mammalian farm animals, mammalian sport animals, and mammalian pets. SLAMF1 ECD fusion molecules incorporating a non-human SLAMF1 ECD are termed “non-human SLAMF1 ECD fusion molecules.” Similar to the human SLAMF1 ECD fusion molecules, non-human fusion molecules may comprise a fusion partner, optional linker, and a SLAMF1 ECD. Such non-human fusion molecules may also include a signal peptide. A “non-human SLAMF1 ECD fragment” refers to a non-human SLAMF1 ECD having one or more residues deleted from the N- and/or C-terminus of the full-length ECD. A “non-human SLAMF1 ECD variant” refers to SLAMF1 ECDs that contain amino acid additions, deletions, and substitutions.

In any of the embodiments described herein, SLAMF1, including but not limited to, full-length SLAMF1, SLAMF1 fragments, SLAMF1 variants, SLAMF1 ECDs, and SLAMF1 ECD fusion proteins, may further comprise a tag. Nonlimiting exemplary tags include FITC, His₆, biotin, and other labels and tags known in the art.

The term “signal peptide” refers to a sequence of amino acid residues located at the N-terminus of a polypeptide that facilitates secretion of a polypeptide from a mammalian cell. A signal peptide may be cleaved upon export of the polypeptide from the mammalian cell, forming a mature protein. Signal peptides may be natural or synthetic, and they may be heterologous or homologous to the protein to which they are attached. Exemplary signal peptides include signal peptides from SLAMF1 and signal peptides from heterologous proteins. A “signal sequence” refers to a polynucleotide sequence that encodes a signal peptide.

The term “vector” is used to describe a polynucleotide that may be engineered to contain a cloned polynucleotide or polynucleotides that may be propagated in a host cell. A vector may include one or more of the following elements: an origin of replication, one or more regulatory sequences (such as, for example, promoters and/or enhancers) that regulate the expression of the polypeptide of interest, and/or one or more selectable marker genes (such as, for example, antibiotic resistance genes and genes that may be used in colorimetric assays, e.g., β-galactosidase). The term “expression vector” refers to a vector that is used to express a polypeptide of interest in a host cell.

A “host cell” refers to a cell that may be or has been a recipient of a vector or isolated polynucleotide. Host cells may be prokaryotic cells or eukaryotic cells. Exemplary eukaryotic cells include mammalian cells, such as primate or non-primate animal cells; fungal cells, such as yeast; plant cells; and insect cells. Nonlimiting exemplary mammalian cells include, but are not limited to, NSO cells, PER.C6® cells (Crucell), and 293 and CHO cells, and their derivatives, such as 293-6E and DG44 cells, respectively.

The term “isolated” as used herein refers to a molecule that has been separated from at least some of the components with which it is typically found in nature or has been separated from at least some of the components with which it is typically produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, e.g., in the case of an RNA polynucleotide. Thus, a DNA polynucleotide that is contained in a vector inside a host cell may be referred to as “isolated” so long as that polynucleotide is not found in that vector in nature.

The terms “subject” and “patient” are used interchangeably herein to refer to a human. In some embodiments, methods of treating other mammals, including, but not limited to, rodents, simians, felines, canines, equines, bovines, porcines, ovines, caprines, mammalian laboratory animals, mammalian farm animals, mammalian sport animals, and mammalian pets, are also provided. In some instances, a “subject” or “patient” refers to a subject or patient in need of treatment for a disease or disorder.

The term “sample” or “patient sample” as used herein, refers to material that is obtained or derived from a subject of interest that contains a cellular and/or other molecular entity that is to be characterized and/or identified, for example based on physical, biochemical, chemical and/or physiological characteristics. For example, the phrase “disease sample” and variations thereof refers to any sample obtained from a subject of interest that would be expected or is known to contain the cellular and/or molecular entity that is to be characterized. By “tissue or cell sample” is meant a collection of similar cells obtained from a tissue of a subject or patient. The source of the tissue or cell sample may be solid tissue as from a fresh, frozen and/or preserved organ or tissue sample or biopsy or aspirate; blood or any blood constituents; bodily fluids such as sputum, cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease tissue/organ. The tissue sample may contain compounds which are not naturally intermixed with the tissue in nature such as preservatives, anticoagulants, buffers, fixatives, nutrients, antibiotics, or the like.

A “reference sample”, “reference cell”, or “reference tissue”, as used herein, refers to a sample, cell or tissue obtained from a source known, or believed, not to be afflicted with the disease or condition for which a method or composition of the invention is being used to identify. In one embodiment, a reference sample, reference cell or reference tissue is obtained from a healthy part of the body of the same subject or patient in whom a disease or condition is being identified using a composition or method of the invention. In one embodiment, a reference sample, reference cell or reference tissue is obtained from a healthy part of the body of at least one individual who is not the subject or patient in whom a disease or condition is being identified using a composition or method of the invention. In some embodiments, a reference sample, reference cell or reference tissue was previously obtained from a patient prior to developing a disease or condition or at an earlier stage of the disease or condition.

A condition “has previously been characterized as having [a characteristic]” when such characteristic of the condition has been shown in at least a subset of patients with the condition, or in one or more animal models of the condition. In some embodiments, such characteristic of the condition does not have to be determined in the patient to be treated one or more SLAMF1 antagonists of the present invention. The presence of the characteristic in a specific patient who is to be treated using the present methods and/or compositions need not have been determined in order for the patient to be considered as having a condition that has previously been characterized as having the characteristic.

A “disorder” or “disease” is any condition that would benefit from treatment with one or more SLAMF1 antagonists of the invention. This includes chronic and acute disorders or diseases including those pathological conditions which predispose the mammal to the disorder in question. Nonlimiting examples of disorders to be treated herein include cancers.

The term “cancer” is used herein to refer to a group of cells that exhibit abnormally high levels of proliferation and growth. A cancer may be benign (also referred to as a benign tumor), pre-malignant, or malignant. Cancer cells may be solid cancer cells or leukemic cancer cells. The term “cancer growth” is used herein to refer to proliferation or growth by a cell or cells that comprise a cancer that leads to a corresponding increase in the size or extent of the cancer.

Examples of cancer include but are not limited to, carcinoma, lymphoma, blastoma, sarcoma, and leukemia. More particular nonlimiting examples of such cancers include squamous cell cancer, small-cell lung cancer, pituitary cancer, esophageal cancer, astrocytoma, soft tissue sarcoma, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, renal cancer, liver cancer, prostate cancer, vulval cancer, thyroid cancer, hepatic carcinoma, brain cancer, endometrial cancer, testis cancer, cholangiocarcinoma, gallbladder carcinoma, gastric cancer, melanoma, and various types of head and neck cancer.

A “chemotherapeutic agent” is a chemical compound useful in the treatment of cancer. Examples of chemotherapeutic agents include, but are not limited to, alkylating agents such as thiotepa and Cytoxan® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Agnew, Chem Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, Adriamycin® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2- ethylhydrazide; procarbazine; PSK® polysaccharide complex (JHS Natural Products, Eugene, OR); razoxane; rhizoxin; sizofiran; spirogermanium; tenuazonic acid; triaziquone; 2,2′, 2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., Taxol® paclitaxel (Bristol- Myers Squibb Oncology, Princeton, N.J.), Abraxane® Cremophor-free, albumin-engineered nanoparticle formulation of paclitaxel (American Pharmaceutical Partners, Schaumberg, Illinois), and Taxotere® doxetaxel (Rhone- Poulenc Rorer, Antony, France); chloranbucil; Gemzar® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; Navelbine® vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluorometlhylornithine (DMFO); retinoids such as retinoic acid; capecitabine; combretastatin; leucovorin (LV); oxaliplatin, including the oxaliplatin treatment regimen (FOLFOX); inhibitors of PKC-alpha, Raf, H-Ras, EGFR (e.g., erlotinib (Tarceva®)) and VEGF-A that reduce cell proliferation and pharmaceutically acceptable salts, acids or derivatives of any of the above.

Further nonlimiting exemplary chemotherapeutic agents include anti-hormonal agents that act to regulate or inhibit hormone action on cancers such as anti-estrogens and selective estrogen receptor modulators (SERMs), including, for example, tamoxifen (including Nolvadex® tamoxifen), raloxifene, droloxifene, 4-hydroxytamoxifen, trioxifene, keoxifene, LY117018, onapristone, and Fareston® toremifene; aromatase inhibitors that inhibit the enzyme aromatase, which regulates estrogen production in the adrenal glands, such as, for example, 4(5)-imidazoles, aminoglutethimide, Megase® megestrol acetate, Aromasin® exemestane, formestanie, fadrozole, Rivisor® vorozole, Femara® letrozole, and Arimidex® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Ralf and H-Ras; ribozymes such as a VEGF expression inhibitor (e.g., Angiozyme® ribozyme) and a HER2 expression inhibitor; vaccines such as gene therapy vaccines, for example, Allovectin® vaccine, Leuvectin® vaccine, and Vaxid® vaccine; Proleukin® rIL-2; Lurtotecan® topoisomerase 1 inhibitor; Abarelix® rmRH; and pharmaceutically acceptable salts, acids or derivatives of any of the above.

An “anti-angiogenesis agent” or “angiogenesis inhibitor” refers to a small molecular weight substance, a polynucleotide (including, e.g., an inhibitory RNA (RNAi or siRNA)), a polypeptide, an isolated protein, a recombinant protein, an antibody, or conjugates or fusion proteins thereof, that inhibits angiogenesis, vasculogenesis, or undesirable vascular permeability, either directly or indirectly. It should be understood that the anti-angiogenesis agent includes those agents that bind and block the angiogenic activity of the angiogenic factor or its receptor. For example, an anti-angiogenesis agent is an antibody or other antagonist to an angiogenic agent, e.g., antibodies to VEGF-A (e.g., bevacizumab (Avastin)) or to the VEGF-A receptor (e.g., KDR receptor or Flt-1 receptor), anti-PDGFR inhibitors such as Gleevec® (Imatinib Mesylate), small molecules that block VEGF receptor signaling (e.g., PTK787/ZK2284, SU6668, Sutent®/SU11248 (sunitinib malate), AMG706, or those described in, e.g., international patent application WO 2004/113304). Anti-angiogensis agents also include native angiogenesis inhibitors , e.g., angiostatin, endostatin, etc. See, e.g., Klagsbrun and D'Amore (1991) Annu. Rev. Physiol. 53:217-39; Streit and Detmar (2003) Oncogene 22:3172-3179 (e.g., Table 3 listing anti-angiogenic therapy in malignant melanoma); Ferrara & Alitalo (1999) Nature Medicine 5(12):1359-1364; Tonini et al. (2003) i Oncogene 22:6549-6556 (e.g., Table 2 listing known anti-angiogenic factors); and, Sato (2003) Int. J. Clin. Oncol. 8:200-206 (e.g., Table 1 listing anti-angiogenic agents used in clinical trials).

A “growth inhibitory agent” as used herein refers to a compound or composition that inhibits growth of a cell (such as a cell expressing VEGF) either in vitro or in vivo. Thus, the growth inhibitory agent may be one that significantly reduces the percentage of cells (such as a cell expressing VEGF) in S phase. Examples of growth inhibitory agents include, but are not limited to, agents that block cell cycle progression (at a place other than S phase), such as agents that induce G1 arrest and M-phase arrest. Classical M-phase blockers include the vincas (vincristine and vinblastine), taxanes, and topoisomerase II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in Mendelsohn and Israel, eds., The Molecular Basis of Cancer, Chapter 1, entitled “Cell cycle regulation, oncogenes, and antineoplastic drugs” by Murakami et al. (W. B. Saunders, Philadelphia, 1995), e.g., p. 13. The taxanes (paclitaxel and docetaxel) are anticancer drugs both derived from the yew tree. Docetaxel (Taxotere®, Rhone-Poulenc Rorer), derived from the European yew, is a semisynthetic analogue of paclitaxel (Taxol®, Bristol-Myers Squibb). Paclitaxel and docetaxel promote the assembly of microtubules from tubulin dimers and stabilize microtubules by preventing depolymerization, which results in the inhibition of mitosis in cells.

The term “anti-neoplastic composition” refers to a composition useful in treating cancer comprising at least one active therapeutic agent. Examples of therapeutic agents include, but are not limited to, e.g., chemotherapeutic agents, growth inhibitory agents, cytotoxic agents, agents used in radiation therapy, anti-angiogenesis agents, cancer immunotherapeutic agents, apoptotic agents, anti-tubulin agents, and other agents to treat cancer, such as anti-HER-2 antibodies, anti-CD20 antibodies, an epidermal growth factor receptor (EGFR) antagonist (e.g., a tyrosine kinase inhibitor), HER1/EGFR inhibitor (e.g., erlotinib (Tarceva®), platelet derived growth factor inhibitors (e.g., Gleevec® (Imatinib Mesylate)), a COX-2 inhibitor (e.g., celecoxib), interferons, CTLA4 inhibitors (e.g., anti-CTLA antibody ipilimumab (YERVOY®)), PD-1 inhibitors (e.g., anti-PD1 antibodies, BMS-936558), PDL1 inhibitors (e.g., anti-PDL1 antibodies, MPDL3280A), PDL2 inhibitors (e.g., anti-PDL2 antibodies), TIM3 inhibitors (e.g., anti-TIM3 antibodies), cytokines, antagonists (e.g., neutralizing antibodies) that bind to one or more of the following targets ErbB2, ErbB3, ErbB4, PDGFR-beta, BlyS, APRIL, BCMA, PD-1, PDL1, PDL2, CTLA4, TIM3, or VEGF receptor(s), TRAIL/Apo2, and other bioactive and organic chemical agents, etc. Combinations thereof are also included in the invention. In some embodiments, an anti-neoplastic composition comprises at least one immunotherapeutic agent, which comprises at least one immune-stimulating agent. In some embodiments, at least one immune stimulating agent comprises an agonist of an immune-stimulatory molecule, including a co-stimulatory molecule. In some embodiments, at least one immune stimulating agent comprises an antagonist of an immune inhibitory molecule, including a co-inhibitory molecule. Additional embodiments involving immune-stimulating agents are discussed below.

“Treatment,” as used herein, covers any administration or application of a therapeutic for a disease (also referred to herein as a “disorder” or a “condition”) in a mammal, including a human, and includes inhibiting the disease or progression of the disease, inhibiting or slowing the disease or its progression, arresting its development, partially or fully relieving the disease, partially or fully relieving one or more symptoms of a disease, or restoring or repairing a lost, missing, or defective function; or stimulating an inefficient process.

The term “effective amount” or “therapeutically effective amount” refers to an amount of a drug effective to treat a disease or disorder in a subject. In some embodiments, an effective amount refers to an amount effective, at dosages and for periods of time necessary, to achieve the desired therapeutic or prophylactic result. A therapeutically effective amount of a SLAMF1 antagonist of the invention may vary according to factors such as the disease state, age, sex, and weight of the individual, and the ability of the antagonist to elicit a desired response in the individual. A therapeutically effective amount encompasses an amount in which any toxic or detrimental effects of a SLAMF1 antagonist are outweighed by the therapeutically beneficial effects.

A “prophylactically effective amount” refers to an amount effective, at dosages and for periods of time necessary, to achieve the desired prophylactic result. Typically, but not necessarily, since a prophylactic dose is used in subjects prior to or at an earlier stage of disease, the prophylactically effective amount would be less than the therapeutically effective amount.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and is compatible with other ingredients of the formulation. The pharmaceutically acceptable carrier is appropriate for the formulation employed. For example, if the therapeutic agent is to be administered orally, the carrier may be a gel capsule. If the therapeutic agent is to be administered subcutaneously, the carrier ideally is not irritable to the skin and does not cause injection site reaction.

An “article of manufacture” is any manufacture (e.g., a package or container) or kit comprising at least one reagent, e.g., a medicament for treatment of a disease or disorder, or a probe for specifically detecting a biomarker described herein. In some embodiments, the manufacture or kit is promoted, distributed, or sold as a unit for performing the methods described herein.

### Therapeutic Compositions and Methods

Methods of Treating Diseases

SLAMF1 antagonists are provided for use in methods of treating humans and other mammals. Methods of treating a disease comprising administering SLAMF1 antagonists to humans and other mammals are provided.

### Methods of Treating Cancer

In some embodiments, methods for treating or preventing a cancer are provided, comprising administering an effective amount of a SLAMF1 antagonist to a subject in need of such treatment.

The present inventors have identified SLAMF1 as a suppressor of activated T cells. SLAMF1 is expressed on activated CD4+and CD8+T cells and it belongs to a family of 9 proteins that have two extracellular immunoglobulin domains and the majority of which have intracellular immunoreceptor tyrosine-based switch motifs (ITSMs). Without intending to be bound by any particular theory, SLAMF1 may interact with T cells, resulting in an inhibitory signal in the T cells that induces an inactive state (e.g., an anergic or tolerized state). Inhibition of SLAMF1 activity, for example, with an inhibitory antibody or soluble SLAMF1, may therefore enhance immune-mediated killing of cancer cells. Targeting molecules include antibodies that bind SLAMF1 and block SLAMF1 activity and SLAMF1 extracellular domain (ECD) and SLAMF1 ECD fusion proteins, including monomeric and dimeric SLAMF1 ECD and SLAMF1 ECD fusion proteins.

In some embodiments, methods of treating cancer are provided, wherein the methods comprise administering a SLAMF1 antagonist to a subject with cancer. In some embodiments, use of a SLAMF1 antagonist for treating cancer is provided. Nonlimiting exemplary cancers that may be treated with SLAMF1 antagonists are provided herein, including carcinoma, lymphoma, blastoma, sarcoma, and leukemia. More particular non-limiting examples of such cancers include squamous cell cancer, small-cell lung cancer, pituitary cancer, esophageal cancer, astrocytoma, soft tissue sarcoma, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, renal cancer, liver cancer, prostate cancer, vulval cancer, thyroid cancer, hepatic carcinoma, brain cancer, endometrial cancer, testis cancer, cholangiocarcinoma, gallbladder carcinoma, gastric cancer, melanoma, and various types of head and neck cancer. In some embodiments, lung cancer is non-small cell lung cancer or lung squamous cell carcinoma. In some embodiments, leukemia is acute myeloid leukemia or chronic lymphocytic leukemia. In some embodiments, breast cancer is breast invasive carcinoma. In some embodiments, ovarian cancer is ovarian serous cystadenocarcinoma. In some embodiments, kidney cancer is kidney renal clear cell carcinoma. In some embodiments, colon cancer is colon adenocarcinoma. In some embodiments, bladder cancer is bladder urothelial carcinoma.

In some embodiments, the SLAMF1 antagonist is a SLAMF1 antibody.

### Methods of Inhibiting Suppression of Activated T Cells

In some embodiments, methods of inhibiting suppression of activated T cells are provided, comprising contacting tissue comprising activated T cells with a SLAMF1 antagonist. In some such embodiments, methods of inhibiting suppression of activated T cells are provided, wherein the activated T cells are suppressed by SLAMF1.

The present inventors have identified SLAMF1 as a suppressor of activated T cells. SLAMF1 is expressed on activated CD4+and CD8+T cells and it belongs to a family of 9 proteins that have two extracellular immunoglobulin domains and the majority of which have intracellular immunoreceptor tyrosine-based switch motifs (ITSMs). Without intending to be bound by any particular theory, SLAMF1 may interact with T cells, resulting in an inhibitory signal in the T cells that induces an inactive state (e.g., an anergic or tolerized state). Inhibition of SLAMF1 activity, for example, with an inhibitory antibody or soluble SLAMF1, may therefore inhibit the suppression of activated T cells by SLAMF1. Targeting molecules include antibodies that bind SLAMF1 and block SLAMF1 activity and SLAMF1 extracellular domain (ECD) and SLAMF1 ECD fusion proteins, including monomeric and dimeric SLAMF1 ECD and SLAMF1 ECD fusion proteins.

In some embodiments, tissue comprising activated T cells is contacted with a SLAMF1 antagonist. In some embodiments, the activated T cells themselves are contacted with a SLAMF1 antagonist. In some embodiments, the activated T cells are in a subject in which the activated T cells are suppressed by SLAMF1.

In some embodiments, the SLAMF1 antagonist is a SLAMF1 antibody.

Routes of Administration and Carriers

In various embodiments, SLAMF1 antagonists may be administered subcutaneously or intravenously. In some embodiments, a SLAMF1 antagonist may be administered in vivo by various routes, including, but not limited to, oral, intra-arterial, parenteral, intranasal, intramuscular, intracardiac, intraventricular, intratracheal, buccal, rectal, intraperitoneal, by inhalation, intradermal, topical, transdermal, and intrathecal, or otherwise, e.g., by implantation. The subject compositions may be formulated into preparations in solid, semi-solid, liquid, or gaseous forms; including, but not limited to, tablets, capsules, powders, granules, ointments, solutions, suppositories, enemas, injections, inhalants, and aerosols. In some embodiments, a SLAMF1 antagonist is delivered using gene therapy. As a nonlimiting example, a nucleic acid molecule encoding a SLAMF1 antagonist may be coated onto gold microparticles and delivered intradermally by a particle bombardment device, or “gene gun,” e.g., as described in the literature (see, e.g., Tang et al., Nature 356:152-154 (1992)).

In various embodiments, compositions comprising a SLAMF1 antagonist are provided in formulations with a wide variety of pharmaceutically acceptable carriers (see, e.g., Gennaro, Remington: The Science and Practice of Pharmacy with Facts and Comparisons: Drugfacts Plus, 20th ed. (2003); Ansel et al., Pharmaceutical Dosage Forms and Drug Delivery Systems, 7^{th} ed., Lippencott Williams and Wilkins (2004); Kibbe et al., Handbook of Pharmaceutical Excipients, 3^{rd} ed., Pharmaceutical Press (2000)). Various pharmaceutically acceptable carriers, which include vehicles, adjuvants, and diluents, are available. Moreover, various pharmaceutically acceptable auxiliary substances, such as pH adjusting and buffering agents, tonicity adjusting agents, stabilizers, wetting agents and the like, are also available. Nonlimiting exemplary carriers include saline, buffered saline, dextrose, water, glycerol, ethanol, and combinations thereof.

In various embodiments, compositions comprising a SLAMF1 antagonist may be formulated for injection, including subcutaneous administration, by dissolving, suspending, or emulsifying them in an aqueous or nonaqueous solvent, such as vegetable or other oils, synthetic aliphatic acid glycerides, esters of higher aliphatic acids, or propylene glycol; and if desired, with conventional additives such as solubilizers, isotonic agents, suspending agents, emulsifying agents, stabilizers and preservatives. In various embodiments, the compositions may be formulated for inhalation, for example, using pressurized acceptable propellants such as dichlorodifluoromethane, propane, nitrogen, and the like. The compositions may also be formulated, in various embodiments, into sustained release microcapsules, such as with biodegradable or non-biodegradable polymers. A nonlimiting exemplary biodegradable formulation includes poly lactic acid-glycolic acid polymer. A nonlimiting exemplary non-biodegradable formulation includes a polyglycerin fatty acid ester. Certain methods of making such formulations are described, for example, in EP 1 125 584 A1.

Pharmaceutical dosage packs comprising one or more containers, each containing one or more doses of a SLAMF1 antagonist, are also provided. In some embodiments, a unit dosage is provided wherein the unit dosage contains a predetermined amount of a composition comprising a SLAMF1 antagonist, with or without one or more additional agents. In some embodiments, such a unit dosage is supplied in single-use prefilled syringe for injection. In various embodiments, the composition contained in the unit dosage may comprise saline, sucrose, or the like; a buffer, such as phosphate, or the like; and/or be formulated within a stable and effective pH range. Alternatively, in some embodiments, the composition may be provided as a lyophilized powder that may be reconstituted upon addition of an appropriate liquid, for example, sterile water. In some embodiments, the composition comprises one or more substances that inhibit protein aggregation, including, but not limited to, sucrose and arginine. In some embodiments, a composition of the invention comprises heparin and/or a proteoglycan.

Pharmaceutical compositions are administered in an amount effective for treatment or prophylaxis of the specific indication. The therapeutically effective amount is typically dependent on the weight of the subject being treated, his or her physical or health condition, the extensiveness of the condition to be treated, or the age of the subject being treated. In some embodiments, a SLAMF1 antagonist may be administered in an amount in the range of about 50 μg/kg body weight to about 50 mg/kg body weight per dose. In some embodiments, a SLAMF1 antagonist may be administered in an amount in the range of about 100 μg/kg body weight to about 50 mg/kg body weight per dose. In some embodiments, a SLAMF1 antagonist may be administered in an amount in the range of about 100 μg/kg body weight to about 20 mg/kg body weight per dose. In some embodiments, a SLAMF1 antagonist may be administered in an amount in the range of about 0.5 mg/kg body weight to about 20 mg/kg body weight per dose.

In some embodiments, a SLAMF1 antagonist may be administered in an amount in the range of about 10 mg to about 1,000 mg per dose. In some embodiments, a SLAMF1 may be administered in an amount in the range of about 20 mg to about 500 mg per dose. In some embodiments, a SLAMF1 antagonist may be administered in an amount in the range of about 20 mg to about 300 mg per dose. In some embodiments, a SLAMF1 antagonist may be administered in an amount in the range of about 20 mg to about 200 mg per dose.

The SLAMF1 antagonist compositions may be administered as needed to subjects. In some embodiments, an effective dose of a SLAMF1 antagonist is administered to a subject one or more times. In various embodiments, an effective dose of a SLAMF1 antagonist is administered to the subject once a month, less than once a month, such as, for example, every two months, every three months, or every six months. In other embodiments, an effective dose of a SLAMF1 antagonist is administered more than once a month, such as, for example, every two weeks, every week, twice per week, three times per week, daily, or multiple times per day. An effective dose of a SLAMF1 antagonist is administered to the subject at least once. In some embodiments, the effective dose of a SLAMF1 antagonist may be administered multiple times, including for periods of at least a month, at least six months, or at least a year. In some embodiments, a SLAMF1 antagonist is administered to a subject as-needed to alleviate one or more symptoms of a condition.

Combination Therapy

An SLAMF1 antagonist according to the invention, including any functional fragments thereof, may be administered to a subject in need thereof in combination with other biologically active substances or other treatment procedures for the treatment of diseases. For example, SLAMF1 antagonists may be administered alone or with other modes of treatment. They may be provided before, substantially contemporaneous with, or after other modes of treatment, such as radiation therapy.

For treatment of cancer, the SLAMF1 antagonist may be administered in conjunction with one or more of anti-cancer agents, such as the chemotherapeutic agent, growth inhibitory agent, anti-angiogenesis agent or anti-neoplastic composition. Nonlimiting examples of chemotherapeutic agent, growth inhibitory agent, anti-angiogenesis agent and anti-neoplastic composition that can be used in combination with one or more SLAMF1 antagonists of the present invention are provided herein under “Definitions.”

In some embodiments, the SLAMF1 antagonist is administered in conjunction with one or more immune-stimulating agents. In some embodiments, at least one immune stimulating agent comprises an agonist of an immune-stimulatory molecule, including a co-stimulatory molecule, while in some embodiments, at least one immune stimulating agent comprises an antagonist of an immune inhibitory molecule, including a co-inhibitory molecule. In some embodiments, at least one immune stimulating agent comprises an agonist of an immune-stimulatory molecule, including a co-stimulatory molecule, found on immune cells, such as T cells. In some embodiments, at least one immune stimulating agent comprises an antagonist of an immune-inhibitory molecule, including a co-inhibitory molecule, found on immune cells, such as T cells. In some embodiments, at least one immune stimulating agent comprises an agonist of an immune-stimulatory molecule, including a co-stimulatory molecule, found on cells involved in innate immunity, such as NK cells. In some embodiments, at least one immune stimulating agent comprises an antagonist of an immune-inhibitory molecule, including a co-inhibitory molecule, found on cells involved in innate immunity, such as NK cells. In some embodiments, the combination enhances the antigen-specific T cell response in the treated subject and/or enhances the innate immunity response in the subject. In some embodiments, the combination results in an improved anti-tumor response in an animal cancer model, such as a xenograft model, compared to administration of either the SLAMF1 antagonist or immune stimulating agent alone. In some embodiments, the combination results in a synergistic response in an animal cancer model, such as a xenograft model, compared to administration of either the SLAMF1 antagonist or immune stimulating agent alone.

In some embodiments, at least one immune stimulating agent comprises an antagonist of an inhibitor of the activation of T cells, while in some embodiments, at least one immune stimulating agent comprises an agonist of a stimulator of the activation of T cells. In some embodiments, at least one immune stimulating agent comprises an antagonist of CTLA4, PD-1, PDL1, PDL2, LAG-3, Galectin 1, Galectin 9, CEACAM-1, BTLA, CD25, CD69, TIGIT, CD113, GPR56, VISTA, B7-H3, B7-H4, 2B4, CD48, GARP, PD1H, LAIR1, TIM1, TIM3, TIM4, ILT4, IL-6, IL-10, TGFβ, VEGF, KIR, adenosine A2A receptor, PI3Kdelta, or IDO. In some embodiments, at least one immune stimulating agent comprises an agonist of B7-1, B7-2, CD28, 4-1BB (CD137), 4-1BBL, ICOS, ICOS-L, OX40, OX4OL, GITR, GITRL, CD27, CD40, CD4OL, DR3, CD28H, IL-2, IL-7, IL-12, IL-15, IL-21, IFNα, STING or a Toll-like receptor agonist such as a TLR2/4 agonist. In some embodiments, at least one immune stimulating agent comprises an agent that binds to a member of the B7 family of membrane-bound proteins such as B7-1, B7-2, B7-H2 (ICOS-L), B7-H3, B7-H4, B7-H5 (VISTA), and B7-H6. In some embodiments, at least one immune stimulating agent comprises an agent that binds to a member of the TNF receptor family or a co-stimulatory or co-inhibitory molecule binding to a member of the TNF receptor family such as CD40, CD4OL, OX40, OX4OL, GITR, GITRL, CD70, CD27L, CD30, CD3OL, 4-1BBL, CD137 (4-1BB), TRAIL/Apo2-L, TRAILR1/DR4, TRAILR2/DR5, TRAILR3, TRAILR4, OPG, RANK, RANKL, TWEAKR/Fn14, TWEAK, BAFFR, EDAR, XEDAR, EDA1, EDA2, TACI, APRIL, BCMA, LTPR, LIGHT, DeR3, HVEM, VEGL/TL1A, TRAMP/DR3, TNFR1, TNFβ, TNFR2, TNFa, 1P2, FAS, FASL, RELT, DR6, TROY, or NGFβ. In some embodiments, at least one immune stimulating agent comprises an agent that antagonizes or inhibits a cytokine that inhibits T cell activation such as IL-6, IL-10, TGFβ, VEGF. In some embodiments, at least one immune stimulating agent comprises an antagonist of a chemokine, such as CXCR2, CXCR4, CCR2, or CCR4. In some embodiments, at least one immune stimulating agent comprises an agonist of a cytokine that stimulates T cell activation such as IL-2, IL-7, IL-12, IL-15, IL-21, and IFNa. In some embodiments, at least one immune stimulating agent comprises an antibody. In some embodiments, at least one immune stimulating agent may comprise a vaccine, such as a mesothelin-targeting vaccine or attenuated listeria cancer vaccine such as CRS-207. Any one or more of the above antagonists, agonists, and binding agents may be combined with any one or more of the anti-SLAMF1 antibodies described herein.

In some embodiments, at least one immune stimulating agent comprises a CD40 agonist, optionally in combination with at least one other immune stimulating agent as listed above. In some embodiments, the CD40 agonist is an antibody. In some embodiments, the CD40 agonist is an anti-CD40 antibody. In some embodiments, the anti-CD40 antibody comprises the CDRs of an antibody selected from CP-870,893; dacetuzumab; SEA-CD40; ADC-1013; RO7009789; and Chi Lob 7/4. In some embodiments, the anti-CD40 antibody comprises the heavy chain and light chain variable regions of an antibody selected from CP-870,893; dacetuzumab; SEA-CD40; ADC-1013; R07009789; and Chi Lob 7/4. In some embodiments, the anti-CD40 antibody is an antibody selected from CP-870,893; dacetuzumab; SEA-CD40; ADC-1013; R07009789; and Chi Lob 7/4. In some embodiments, the CD40 agonist is recombinant CD4OL. In some embodiments, at least one immune stimulating agent comprises a CD40 agonist and at least one additional immune stimulating agent from any of those described above. For example, any one or more of the above immune stimulating agents above may be combined with any one or more of the SLAMF1 antagonists described herein as well as with a CD40 agonist, such as a CD40 agonist antibody or recombinant CD4OL, such as any one of the anti-CD40 antibodies described above.

In some embodiments, the SLAMF1 antagonist and at least one immune stimulatory agent are administered concurrently or sequentially. In some embodiments, the SLAMF1 antagonist and at least one immune stimulatory agent are administered concurrently. In some embodiments, one or more doses of at least one immune stimulatory agent are administered prior to administering an SLAMF1 antagonist. In some embodiments, the subject received a complete course of therapy with at least one immune stimulatory agent prior to administration of the SLAMF1 antagonist. In some embodiments, the SLAMF1 antagonist is administered during a second course of therapy with at least one immune stimulatory agent. In some embodiments, the subject received at least one, at least two, at least three, or at least four doses of at least one immune stimulatory agent prior to administration of the SLAMF1 antagonist. In some embodiments, at least one dose of at least one immune stimulatory agent is administered concurrently with the SLAMF1 antagonist. In some embodiments, one or more doses of the SLAMF1 antagonist are administered prior to administering at least one immune stimulatory agent. In some embodiments, the subject received at least two, at least three, or at least four doses of the SLAMF1 antagonist prior to administration of at least one immune stimulatory agent. In some embodiments, at least one dose of the SLAMF1 antagonist is administered concurrently with the at least one immune stimulatory agent.

In some embodiments, compositions are provided, comprising an SLAMF1 antagonist and at least one immune stimulatory agent. In some embodiments, at least one immune stimulating agent comprises an antagonist of an inhibitor of the activation of T cells, while in some embodiments, at least one immune stimulating agent comprises comprises an agonist of a stimulator of the activation of T cells. In some embodiments, at least one immune stimulating agent comprises an antagonist of CTLA4, PD-1, PDL1, PDL2, LAG-3, Galectin 1, Galectin 9, CEACAM-1, BTLA, CD25, CD69, TIGIT, CD113, GPR56, VISTA, B7-H3, B7-H4, 2B4, CD48, GARP, PD1H, LAIR1, TIM1, TIM3, TIM4, ILT4, IL-6, IL-10, TGFβ, VEGF, KIR, adenosine A2A receptor, PI3Kdelta, or IDO. In some embodiments, at least one immune stimulating agent comprises an agonist of B7-1, B7-2, CD28, 4-1BB (CD137), 4-1BBL, ICOS, ICOS-L, OX40, OX4OL, GITR, GITRL, CD27, CD40, CD4OL, DR3, CD28H, IL-2, IL-7, IL-12, IL-15, IL-21, IFNα, STING, or a Toll-like receptor agonist such as a TLR2/4 agonist. In some embodiments, at least one immune stimulating agent comprises an agent that binds to a member of the B7 family of membrane-bound proteins such as B7-1, B7-2, B7-H2 (ICOS-L), B7-H3, B7-H4, B7-H5 (VISTA), and B7-H6. In some embodiments, at least one immune stimulating agent comprises an agent that binds to a member of the TNF receptor family or a co-stimulatory or co-inhibitory molecule binding to a member of the TNF receptor family such as CD40, CD4OL, OX40, OX4OL, GITR, GITRL, CD70, CD27L, CD30, CD3OL, 4-1BBL, CD137 (4-1BB), TRAIL/Apo2-L, TRAILR1/DR4, TRAILR2/DR5, TRAILR3, TRAILR4, OPG, RANK, RANKL, TWEAKR/Fn14, TWEAK, BAFFR, EDAR, XEDAR, EDA1, EDA2, TACI, APRIL, BCMA, LTPR, LIGHT, DeR3, HVEM, VEGL/TL1A, TRAMP/DR3, TNFR1, TNFβ, TNFR2, TNFα, 1β2, FAS, FASL, RELT, DR6, TROY, or NGFβ. In some embodiments, at least one immune stimulating agent comprises an agent that antagonizes or inhibits a cytokine that inhibits T cell activation such as IL-6, IL-10, TGFβ, VEGF. In some embodiments, at least one immune stimulating agent comprises an agonist of a cytokine that stimulates T cell activation such as IL-2, IL-7, IL-12, IL-15, IL-21, and IFNα. In some embodiments, at least one immune stimulating agent comprises an antagonist of a chemokine, such as CXCR2, CXCR4, CCR2, or CCR4. In some embodiments, at least one immune stimulating agent comprises an antibody. In some embodiments, at least one immune stimulating agent may comprise a vaccine, such as a mesothelin-targeting vaccine or attenuated listeria cancer vaccine such as CRS-207.

In some embodiments, the compositions comprise any one or more of the above antagonists, agonists, and binding agents combined with any one or more of the SLAMF1 antagonists described herein. The compositions may include each therapeutic agent in a separate container or compartment or alternatively, may include two or more of the therapeutic agents mixed together.

### SLAMF1 Antibodies

In some embodiments, antibodies that inhibit SLAMF1 activity are provided. In some embodiments, the SLAMF1 activity is SLAMF1-mediated suppression of activated T cells. In some such embodiments, the antibody is a SLAMF1 antibody. In some embodiments, a SLAMF1 antibody binds to SLAMF1 extracellular domain (ECD). In some embodiments, a SLAMF1 antibody inhibits SLAMF1-mediated signaling.

In some embodiments, a SLAMF1 antibody has a dissociation constant (Kd) of ≦1 μM, ≦100 nM, ≦10 nM, ≦1 nM, ≦0.1 nM, ≦0.01 nM, or ≦0.001 nM (e.g. 10⁻⁸ M or less, e.g. from 10⁻⁸ M to 10⁻¹³ M, e.g., from 10⁻⁹M to 10⁻¹³ M) for SLAMF1.

In some embodiments, an antibody binds to SLAMF1 from multiple species. For example, in some embodiments, an antibody binds to human SLAMF1, and also binds to SLAMF1 from at least one mammal selected from mouse, rat, dog, guinea pig, and monkey.

In some embodiments, multispecific antibodies are provided. In some embodiments, bispecific antibodies are provided. Nonlimiting exemplary bispecific antibodies include antibodies comprising a first arm comprising a heavy chain/light chain combination that binds a first antigen and a second arm comprising a heavy chain/light chain combination that binds a second antigen. A further nonlimiting exemplary multispecific antibody is a dual variable domain antibody. In some embodiments, a bispecific antibody comprises a first arm that inhibits SLAMF1 activity and a second arm that stimulates T cells. In some embodiments, the second arm binds PD-1 or PD-Ll. In some embodiments, the first arm binds SLAMF1.

In some embodiments, single chain antibodies that inhibit SLAMF1 activity are provided, such as camelid antibodies.

In some embodiments, fibronectin type III domain antibodies that inhibit SLAMF1 activity are provided, such as Adnectins™.

Humanized Antibodies

In some embodiments, a SLAMF1 antibody is a humanized antibody. Humanized antibodies are useful as therapeutic molecules because humanized antibodies reduce or eliminate the human immune response to non-human antibodies (such as the human anti-mouse antibody (HAMA) response), which can result in an immune response to an antibody therapeutic, and decreased effectiveness of the therapeutic.

An antibody may be humanized by any method. Nonlimiting exemplary methods of humanization include methods described, e.g., in U.S. Pat. Nos. 5,530,101; 5,585,089; 5,693,761; 5,693,762; 6,180,370; Jones et al., Nature 321: 522-525 (1986); Riechmann et al., Nature 332: 323-27 (1988); Verhoeyen et al., Science 239: 1534-36 (1988); and U.S. Publication No. US 2009/0136500.

As noted above, a humanized antibody is an antibody in which at least one amino acid in a framework region of a non-human variable region has been replaced with the amino acid from the corresponding location in a human framework region. In some embodiments, at least two, at least three, at least four, at least five, at least six, at least seven, at least eight, at least nine, at least 10, at least 11, at least 12, at least 15, or at least 20 amino acids in the framework regions of a non-human variable region are replaced with an amino acid from one or more corresponding locations in one or more human framework regions.

In some embodiments, some of the corresponding human amino acids used for substitution are from the framework regions of different human immunoglobulin genes. That is, in some such embodiments, one or more of the non-human amino acids may be replaced with corresponding amino acids from a human framework region of a first human antibody or encoded by a first human immunoglobulin gene, one or more of the non-human amino acids may be replaced with corresponding amino acids from a human framework region of a second human antibody or encoded by a second human immunoglobulin gene, one or more of the non-human amino acids may be replaced with corresponding amino acids from a human framework region of a third human antibody or encoded by a third human immunoglobulin gene, etc. Further, in some embodiments, all of the corresponding human amino acids being used for substitution in a single framework region, for example, FR2, need not be from the same human framework. In some embodiments, however, all of the corresponding human amino acids being used for substitution are from the same human antibody or encoded by the same human immunoglobulin gene.

In some embodiments, an antibody is humanized by replacing one or more entire framework regions with corresponding human framework regions. In some embodiments, a human framework region is selected that has the highest level of homology to the non-human framework region being replaced. In some embodiments, such a humanized antibody is a CDR-grafted antibody.

In some embodiments, following CDR-grafting, one or more framework amino acids are changed back to the corresponding amino acid in a mouse framework region. Such “back mutations” are made, in some embodiments, to retain one or more mouse framework amino acids that appear to contribute to the structure of one or more of the CDRs and/or that may be involved in antigen contacts and/or appear to be involved in the overall structural integrity of the antibody. In some embodiments, ten or fewer, nine or fewer, eight or fewer, seven or fewer, six or fewer, five or fewer, four or fewer, three or fewer, two or fewer, one, or zero back mutations are made to the framework regions of an antibody following CDR grafting.

In some embodiments, a humanized antibody also comprises a human heavy chain constant region and/or a human light chain constant region.

Chimeric Antibodies

In some embodiments, a SLAMF1 antibody is a chimeric antibody. In some embodiments, a SLAMF1 antibody comprises at least one non-human variable region and at least one human constant region. In some such embodiments, all of the variable regions of a SLAMF1 antibody are non-human variable regions, and all of the constant regions of the SLAMF1 antibody are human constant regions. In some embodiments, one or more variable regions of a chimeric antibody are mouse variable regions. The human constant region of a chimeric antibody need not be of the same isotype as the non-human constant region, if any, it replaces. Chimeric antibodies are discussed, e.g., in U.S. Pat. No. 4,816,567; and Morrison et al. Proc. Natl. Acad. Sci. USA 81: 6851-55 (1984).

Human Antibodies

In some embodiments, a SLAMF1 antibody is a human antibody. Human antibodies can be made by any suitable method. Nonlimiting exemplary methods include making human antibodies in transgenic mice that comprise human immunoglobulin loci. See, e.g., Jakobovits et al., Proc. Natl. Acad. Sci. USA 90: 2551-55 (1993); Jakobovits et al., Nature 362: 255-8 (1993); Lonberg et al., Nature 368: 856-9 (1994); and U.S. Pat. Nos. 5,545,807; 6,713,610; 6,673,986; 6,162,963; 5,545,807; 6,300,129; 6,255,458; 5,877,397; 5,874,299; and 5,545,806.

Nonlimiting exemplary methods also include making human antibodies using phage display libraries. See, e.g., Hoogenboom et al., J. Mol. Biol. 227: 381-8 (1992); Marks et al., J. Mol. Biol. 222: 581-97 (1991); and PCT Publication No. WO 99/10494.

Human Antibody Constant Regions

In some embodiments, a humanized, chimeric, or human antibody described herein comprises one or more human constant regions. In some embodiments, the human heavy chain constant region is of an isotype selected from IgA, IgG, and IgD. In some embodiments, the human light chain constant region is of an isotype selected from lc and 2L In some embodiments, an antibody described herein comprises a human IgG constant region, for example, human IgG1, IgG2, IgG3, or IgG4. In some embodiments, an antibody or Fc fusion partner comprises a C237S mutation, for example, in an IgG1 constant region. See, e.g., SEQ ID NO: 6. In some embodiments, an antibody described herein comprises a human IgG2 heavy chain constant region. In some such embodiments, the IgG2 constant region comprises a P331S mutation, as described in U.S. Pat. No. 6,900,292. In some embodiments, an antibody described herein comprises a human IgG4 heavy chain constant region. In some such embodiments, an antibody described herein comprises an S241P mutation in the human IgG4 constant region. See, e.g., Angal et al. Mol. Immunol. 30(1): 105-108 (1993). In some embodiments, an antibody described herein comprises a human IgG4 constant region and a human κ light chain.

The choice of heavy chain constant region can determine whether or not an antibody will have effector function in vivo. Such effector function, in some embodiments, includes antibody-dependent cell-mediated cytotoxicity (ADCC) and/or complement-dependent cytotoxicity (CDC), and can result in killing of the cell to which the antibody is bound. Typically, antibodies comprising human IgG1 or IgG3 heavy chains have effector function.

In some embodiments, effector function is not desirable. For example, in some embodiments, effector function may not be desirable in treatments of inflammatory conditions and/or autoimmune disorders. In some such embodiments, a human IgG4 or IgG2 heavy chain constant region is selected or engineered. In some embodiments, an IgG4 constant region comprises an S241P mutation.

### Exemplary Properties of Antibodies

Exemplary Properties of SLAMF1 Antibodies

In some embodiments, a SLAMF1 antibody binds to SLAMF1 and inhibits SLAMF1-mediated signaling. In some embodiments, a SLAMF1 antibody inhibits SLAMF1-mediated suppression of activated T cells. In some embodiments, a SLAMF1 antibody inhibits SLAMF1-mediated suppression of activated CD3+T cells. In some embodiments, a SLAMF1 antibody inhibits SLAMF1-mediated suppression of IL-2-activated CD3+T cells. In some embodiments, a SLAMF1 antibody binds to SLAMF1 with a binding affinity (KD) of less than 50 nM, less than 20 nM, less than 10 nM, or less than 1 nM. In some embodiments, the extent of binding of a SLAMF1 antibody to an unrelated, non-SLAMF1 protein is less than about 10% of the binding of the antibody to SLAMF1 as measured, e.g., by a radioimmunoassay (RIA). In some embodiments, a SLAMF1 antibody binds to an epitope of SLAMF1 that is conserved among SLAMF1 from different species. In some embodiments, a SLAMF1 antibody binds to the same epitope as a human or humanized SLAMF1 antibody that binds human SLAMF1.

### Antibody Conjugates

In some embodiments, a SLAMF1 is conjugated to a label. As used herein, a label is a moiety that facilitates detection of the antibody and/or facilitates detection of a molecule to which the antibody binds. Nonlimiting exemplary labels include, but are not limited to, radioisotopes, fluorescent groups, enzymatic groups, chemiluminescent groups, biotin, epitope tags, metal-binding tags, etc. One skilled in the art can select a suitable label according to the intended application.

In some embodiments, a label is conjugated to an antibody using chemical methods in vitro. Nonlimiting exemplary chemical methods of conjugation are known in the art, and include services, methods and/or reagents commercially available from, e.g., Thermo Scientific Life Science Research Produces (formerly Pierce; Rockford, Ill.), Prozyme (Hayward, Calif.), SACRI Antibody Services (Calgary, Canada), AbD Serotec (Raleigh, N.C.), etc. In some embodiments, when a label is a polypeptide, the label can be expressed from the same expression vector with at least one antibody chain to produce a polypeptide comprising the label fused to an antibody chain.

### Signal Peptides

In order for some secreted proteins to express and secrete in large quantities, a signal peptide from a heterologous protein may be desirable. Employing heterologous signal peptides may be advantageous in that a resulting mature polypeptide may remain unaltered as the signal peptide is removed in the ER during the secretion process. The addition of a heterologous signal peptide may be required to express and secrete some proteins.

Nonlimiting exemplary signal peptide sequences are described, e.g., in the online Signal Peptide Database maintained by the Department of Biochemistry, National University of Singapore. See Choo et al., BMC Bioinformatics, 6: 249 (2005); and PCT Publication No. WO 2006/081430.

### Co-Translational and Post-Translational Modifications

In some embodiments, a polypeptide such as a SLAMF1 is differentially modified during or after translation, for example by glycosylation, sialylation, acetylation, phosphorylation, amidation, derivatization by known protecting/blocking groups, proteolytic cleavage, or linkage to an antibody molecule or other cellular ligand. Any of numerous chemical modifications may be carried out by known techniques, including, but not limited to, specific chemical cleavage by cyanogen bromide, trypsin, chymotrypsin, papain, V8 protease; NABH₄, acetylation; formylation; oxidation; reduction; and/or metabolic synthesis in the presence of tunicamycin.

Additional post-translational modifications encompassed by the invention include, for example, N-linked or O-linked carbohydrate chains; processing of N-terminal or C-terminal ends; attachment of chemical moieties to the amino acid backbone; chemical modifications of N-linked or O-linked carbohydrate chains; and addition or deletion of an N-terminal methionine residue as a result of prokaryotic host cell expression.

### Nucleic Acid Molecules Encoding SLAMF1 Antagonists

Nucleic acid molecules are provided, wherein the nucleic acid molecules comprise polynucleotides that encode one or more chains of an antibody described herein, such as a SLAMF1. In some embodiments, a nucleic acid molecule comprises a polynucleotide that encodes a heavy chain or a light chain of an antibody described herein. In some embodiments, a nucleic acid molecule comprises both a polynucleotide that encodes a heavy chain and a polynucleotide that encodes a light chain, of an antibody described herein. In some embodiments, a first nucleic acid molecule comprises a first polynucleotide that encodes a heavy chain and a second nucleic acid molecule comprises a second polynucleotide that encodes a light chain.

In some such embodiments, the heavy chain and the light chain are expressed from one nucleic acid molecule, or from two separate nucleic acid molecules, as two separate polypeptides. In some embodiments, such as when an antibody is an scFv, a single polynucleotide encodes a single polypeptide comprising both a heavy chain and a light chain linked together.

In some embodiments, a polynucleotide encoding a heavy chain or light chain of an antibody described herein comprises a nucleotide sequence that encodes a leader sequence, which, when translated, is located at the N-terminus of the heavy chain or light chain. As discussed above, the leader sequence may be the native heavy or light chain leader sequence, or may be another heterologous leader sequence.

Nucleic acid molecules may be constructed using recombinant DNA techniques conventional in the art. In some embodiments, a nucleic acid molecule is an expression vector that is suitable for expression in a selected host cell.

### Polypeptide Expression and Production

Vectors

Vectors comprising polynucleotides that encode heavy chains and/or light chains of the antibodies described herein are provided. Such vectors include, but are not limited to, DNA vectors, phage vectors, viral vectors, retroviral vectors, etc. In some embodiments, a vector comprises a first polynucleotide sequence encoding a heavy chain and a second polynucleotide sequence encoding a light chain. In some embodiments, the heavy chain and light chain are expressed from the vector as two separate polypeptides. In some embodiments, the heavy chain and light chain are expressed as part of a single polypeptide, such as, for example, when the antibody is an scFv.

In some embodiments, a first vector comprises a polynucleotide that encodes a heavy chain and a second vector comprises a polynucleotide that encodes a light chain. In some embodiments, the first vector and second vector are transfected into host cells in similar amounts (such as similar molar amounts or similar mass amounts). In some embodiments, a mole- or mass-ratio of between 5:1 and 1:5 of the first vector and the second vector is transfected into host cells. In some embodiments, a mass ratio of between 1:1 and 1:5 for the vector encoding the heavy chain and the vector encoding the light chain is used. In some embodiments, a mass ratio of 1:2 for the vector encoding the heavy chain and the vector encoding the light chain is used.

In some embodiments, a vector is selected that is optimized for expression of polypeptides in CHO or CHO-derived cells, or in NSO cells. Exemplary such vectors are described, e.g., in Running Deer et al., Biotechnol. Prog. 20:880-889 (2004).

In some embodiments, a vector is chosen for in vivo expression of a SLAMF1 antagonist in animals, including humans. In some such embodiments, expression of the polypeptide or polypeptides is under the control of a promoter or promoters that function in a tissue-specific manner. For example, liver-specific promoters are described, e.g., in PCT Publication No. WO 2006/076288.

Host Cells

In various embodiments, heavy chains and/or light chains of the antibodies described herein may be expressed in prokaryotic cells, such as bacterial cells; or in eukaryotic cells, such as fungal cells (such as yeast), plant cells, insect cells, and mammalian cells. Such expression may be carried out, for example, according to procedures known in the art. Exemplary eukaryotic cells that may be used to express polypeptides include, but are not limited to, COS cells, including COS 7 cells; 293 cells, including 293-6E cells; CHO cells, including CHO-S and DG44 cells; PER.C6® cells (Crucell); and NSO cells. In some embodiments, heavy chains and/or light chains of the antibodies described herein may be expressed in yeast. See, e.g., U.S. Publication No. US 2006/0270045 Al. In some embodiments, a particular eukaryotic host cell is selected based on its ability to make desired post-translational modifications to the heavy chains and/or light chains of a SLAMF1 antibody. For example, in some embodiments, CHO cells produce polypeptides that have a higher level of sialylation than the same polypeptide produced in 293 cells.

Introduction of one or more nucleic acids into a desired host cell may be accomplished by any method, including but not limited to, calcium phosphate transfection, DEAE-dextran mediated transfection, cationic lipid-mediated transfection, electroporation, transduction, infection, etc. Nonlimiting exemplary methods are described, e.g., in Sambrook et al., Molecular Cloning, A Laboratory Manual, 3’ ed. Cold Spring Harbor Laboratory Press (2001). Nucleic acids may be transiently or stably transfected in the desired host cells, according to any suitable method.

In some embodiments, one or more polypeptides may be produced in vivo in an animal that has been engineered or transfected with one or more nucleic acid molecules encoding the polypeptides, according to any suitable method.

Purification of Polypeptides

The antibodies described herein may be purified by any suitable method. Such methods include, but are not limited to, the use of affinity matrices or hydrophobic interaction chromatography. Suitable affinity ligands include the antigen and/or epitope to which the antibody binds, and ligands that bind antibody constant regions. For example, a Protein A, Protein G, Protein A/G, or an antibody affinity column may be used to bind the constant region and to purify an antibody.

In some embodiments, hydrophobic interactive chromatography, for example, a butyl or phenyl column, is also used for purifying some polypeptides. Many methods of purifying polypeptides are known in the art.

Cell-free Production of Polypeptides

In some embodiments, an antibody described herein is produced in a cell-free system. Nonlimiting exemplary cell-free systems are described, e.g., in Sitaraman et al., Methods Mol. Biol. 498: 229-44 (2009); Spirin, Trends Biotechnol. 22: 538-45 (2004); Endo et al., Biotechnol. Adv. 21: 695-713 (2003).

### Methods of Identifying SLAMF1 Antagonists

In some embodiments, methods of identifying SLAMF1 antagonists are provided. In some embodiments, a method comprises contacting a candidate molecule with a SLAMF1, a SLAMF1 ECD, or a SLAMF1 ECD fusion molecule (collectively referred to as an “SLAMF1 molecule”). In some embodiments, the method further comprises contacting IL-2-activated CD3+T cells with the candidate molecule / SLAMF1 molecule mixture and determining the effect on T cell activation. In some embodiments, the method comprises contacting IL-2-activated CD3+T cells with the candidate molecule and then contacting the mixture with a SLAMF1 molecule, and determining the effect on T cell activation. In some embodiments, the assay is carried out substantially as described in Example 3 herein, but in the presence of a candidate molecule. In some embodiments, if suppression of T cell activation is reduced in the presence of the candidate molecule relative to suppression of T cell activation in the presence of the SLAMF1 molecule alone, the candidate molecule is a SLAMF1 antagonist. In some embodiments, the candidate molecule reduces suppression of T cell activation by at least 20%, at least 30%, at least 40%, at least 50%, at least 60%, at least 70%, at least 80%, or at least 90%. In some embodiments, the candidate molecule is an anti-SLAMF1 antibody. One skilled in the art will recognize that the order in which the components are contacted with one another may be varied according to the assay design.

In some embodiments, the SLAMF1 molecule is a full length SLAMF1, for example, SLAMF1 expressed on the surface of a cell. In some embodiments, the SLAMF1 molecule is a soluble SLAMF1, such as a SLAMF1 ECD or SLAMF1 ECD fusion molecule.

Exemplary classes of candidate molecules include, but are not limited to, antibodies, peptides, small molecules, and aptamers. In some embodiments, a candidate molecule is an antibody that is known to bind to SLAMF1 (i.e., a SLAMF1 antibody).

### Articles of Manufacture

In some embodiments, an article of manufacture or a kit containing materials useful for the detection of a biomarker (e.g., SLAMF1) or for the treatment, prevention and/or diagnosis of the disorders described above is provided. The article of manufacture comprises a container and a label or package insert on or associated with the container. Suitable containers include, for example, bottles, vials, syringes, etc. The containers may be formed from a variety of materials such as glass or plastic. In some embodiments, the container holds a composition which is by itself or combined with another composition effective for treating, preventing and/or diagnosing the condition and may have a sterile access port (for example the container may be an intravenous solution bag or a vial having a stopper pierceable by a hypodermic injection needle). The label or package insert indicates that the composition is used for treating the condition of choice. In some embodiments, the article of manufacture may comprise (a) a first container with a composition contained therein, wherein the composition comprises a SLAMF1 antagonist of the invention; and (b) a second container with a composition contained therein, wherein the composition comprises an additional therapeutic agent. The article of manufacture may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

In some embodiments, the molecules of the present invention can be packaged alone or in combination with other therapeutic compounds as a kit. In one embodiment, the therapeutic compound is an anti-cancer agent. In another embodiment, the therapeutic compound is an immunosuppressive agent. The kit can include optional components that aid in the administration of the unit dose to patients, such as vials for reconstituting powder forms, syringes for injection, customized IV delivery systems, inhalers, etc. Additionally, the unit dose kit can contain instructions for preparation and administration of the compositions. The kit may be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like.

### EXAMPLES

The examples discussed below are intended to be purely exemplary of the invention and should not be considered to limit the invention in any way. The examples are not intended to represent that the experiments below are all or the only experiments performed. Efforts have been made to ensure accuracy with respect to numbers used (for example, amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1:

### Materials and Methods

Cells/blood. All buffy coats were obtained from Stanford Blood Center.

CD3⁺T cell enrichment. PBMCs were enriched from buffy coat using a ficoll gradient. Total CD3^{|} T cells were negatively enriched using the EasySep™ Human T Cell Enrichment Kit from StemCell Technologies based on manufacturer's instructions.

CD3⁺ T cell activation and IL-2 rest. Enriched CD3⁺ T cells were activated with anti-CD3/anti-CD28 Dynabeads (Life Technologies) at a 1:1 cell to bead ratio and a 2×10⁵ cell/mL concentration for six days at 37° C. Beads were removed from cells using a Dynal magnet (Life Technologies) and cultured in the presence of 10U/mL IL-2 (R&D Systems) at a cell concentration of lx10⁶cells/mL for an additional four days at 37° C. Cells were thoroughly washed to remove IL-2 and used in proliferation assays

Proliferation assay. 96-well tissue culture treated plates were coated with 1.35μg/mL anti-human CD3 (clone OKT3, eBioscience) and 20μg/mL anti-human IgG (Jackson ImmunoResearch) in 1×PBS overnight at 4° C. Plates were thoroughly washed with 1×PBS to remove free protein and re-coated at 37° C. for 4hours with titrating doses of Fc-proteins (starting at 100μg/mL; 1:3 dilutions in lx PBS). Human IgG1 and human PD-L1-hIgG1 negative and positive controls were purchased from R&D Systems, whereas human SLAMF1-hIgG1 was produced at Five Prime Therapeutics. Following re-coating of Fc-proteins, the plates were thoroughly washed with lx PBS to remove free protein and activated/IL-2 rested CD3⁺T cells were added to the plates at a lx10⁶ cells/mL concentration. The cells are incubated on the plates for 72hrs at 37° C. 12-16 hours before harvesting, cells are pulsed with 5μM Edu (Life Technologies) and incubated at 37° C. Percent proliferating cells are measured by flow cytometry on a LSRII (BD Biosciences) using the Click-iT Plus Edu Flow Cytometry Assay kit (Life Technologies) according to manufacturer's instructions. Data was analyzed using FlowJo software.

### Example 2:

### PD-L1 suppresses proliferation of activated/IL-2 rested CD3⁺ T cells

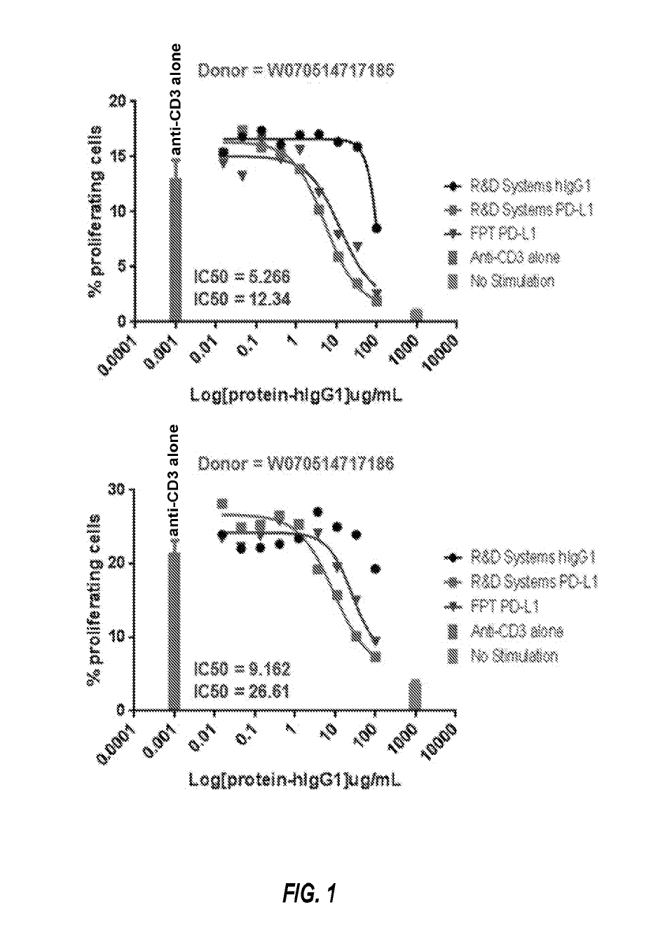

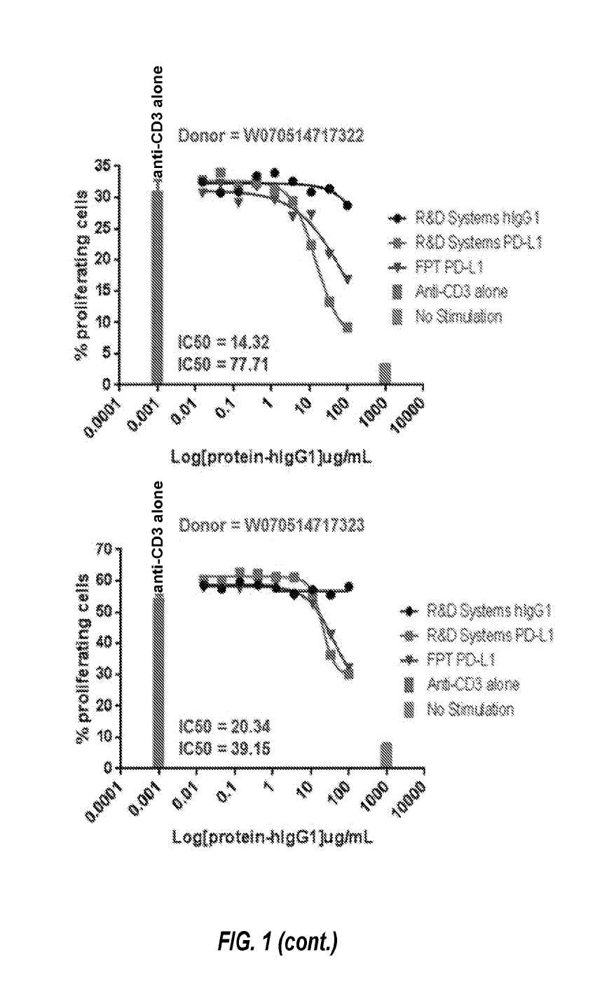

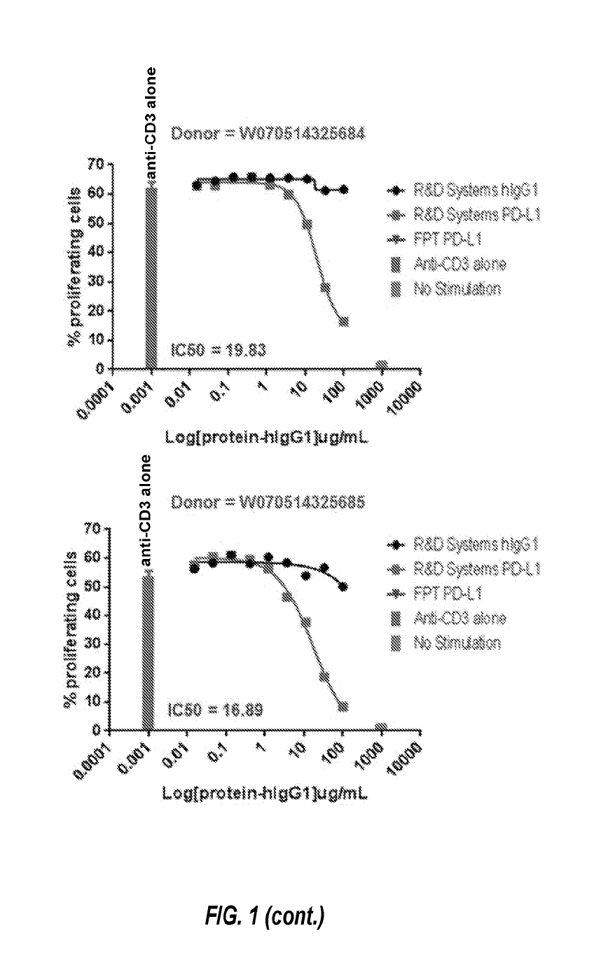

To explore whether this immobilized Fc-protein assay format was able to identify proteins capable of suppressing proliferation of T cells, CD3^{|} T cells that had been previously activated and rested in IL-2 were added to plates that had been coated with anti-CD3, anti-human IgG and either hIgG1 and PD-L1 from R&D systems or PD-L1 produced at Five Prime Therapeutics. 72 hour re-stimulation of activated/IL-2 rested T cells in the presence of these Fc-proteins demonstrated that only in the presence of PD-Ll was proliferation suppressed (FIG. 1). Whereas dose dependent suppression was observed in the presence of either PD-L1 protein, no dose dependent suppression was observed in the presence of hIgG1. There was some suppression at the highest concentration of hIgG1 tested (i.e. 100μg/mL) suggesting that concentrations >100μg/mL may yield some non-specific suppression. This data demonstrates that this assay system is capable of identifying Fc-proteins capable of suppressing proliferation of T cells.

### Example 3:

### SLAMF1-Fc suppresses proliferation of activated/IL-2 rested CD3⁺ T cells

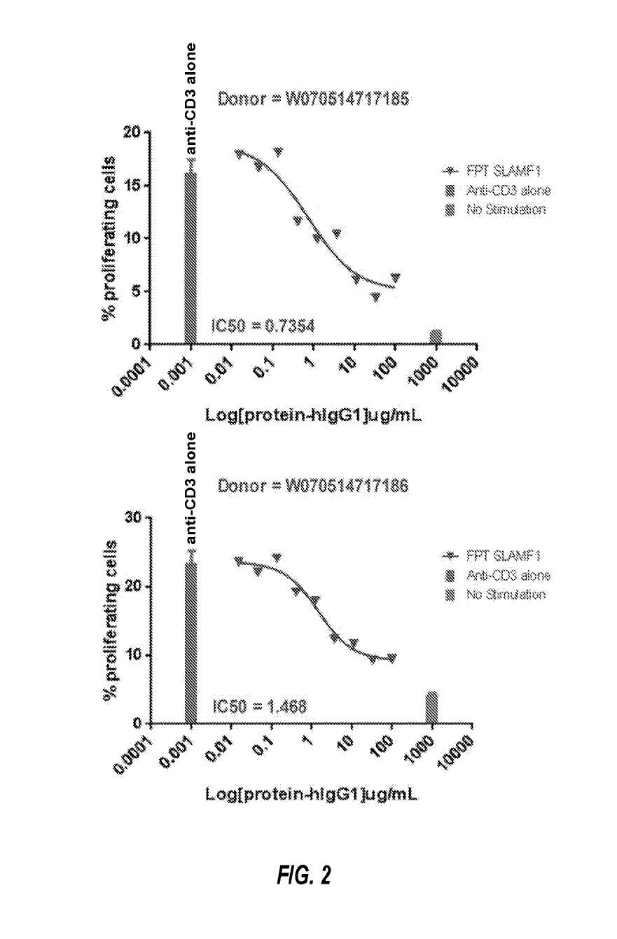

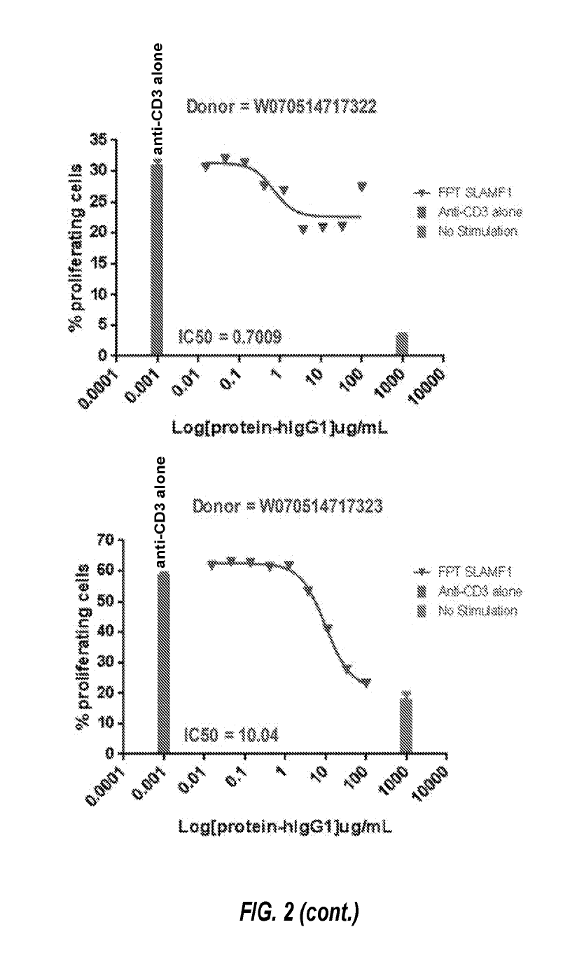

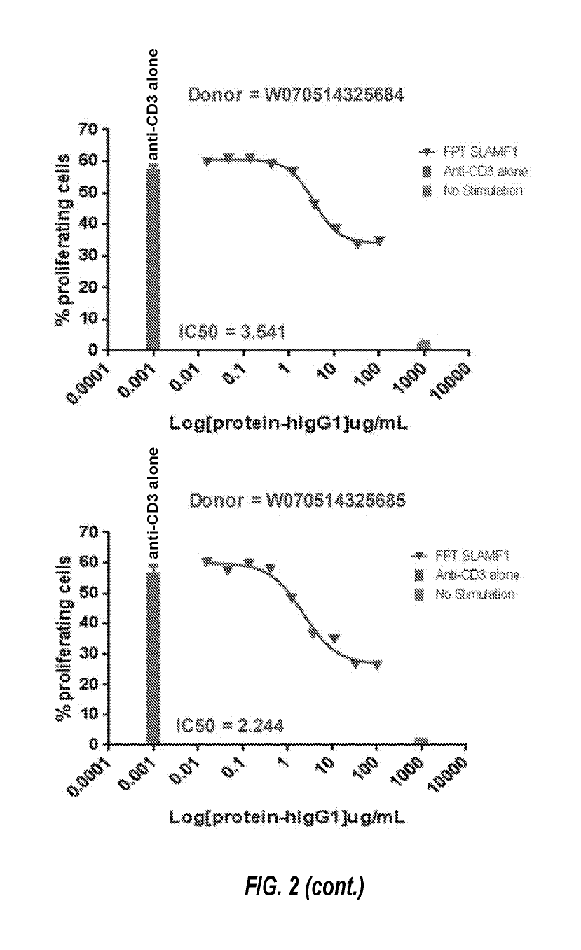

To explore whether SLAMF1 ECD-Fc would suppress proliferation of T cells in this purified T cell assay format, CD3+T cells that had been previously activated and rested in IL-2 were again added to plates that had been coated with anti-CD3, anti-human IgG and SLAMF1-Fc, produced at Five Prime Therapeutics. 72 hour re-stimulation of activated/IL-2 rested T cells in the presence of SLAMF1-Fc demonstrated that SLAMF1-Fc suppressed proliferation in a dose dependent manner (FIG. 2). SLAMF1-Fc demonstrated close to full inhibition in 3 donors tested and partial inhibition in 3 donors tested. These data suggest that SLAMF1-Fc suppresses proliferation of T cells.

### Example 4:

### SLAMF1 Expression in Human Tissues

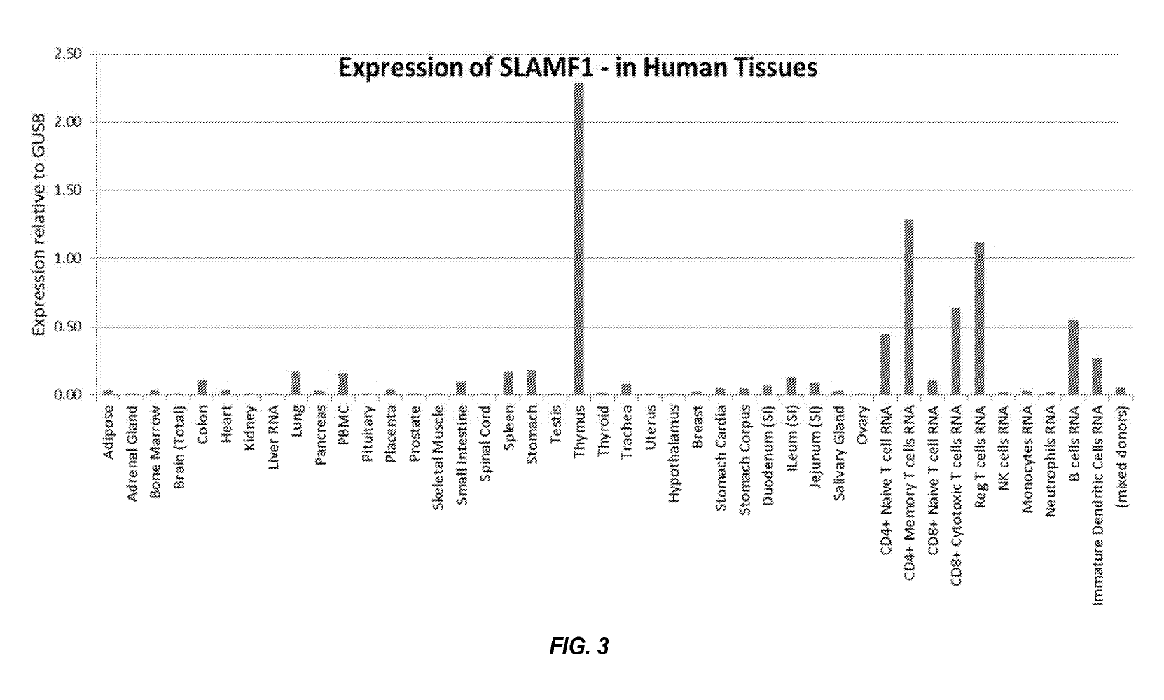

SLAMF1 was originally identified as being expressed exclusively in lymphoid cells by RT-PCR. See Cocks et al., 1995, Nature 376: 260-263. RT-PCR was performed on a panel of human tissue RNA (Clontech) and human immune cell RNA (AllCells, LLC), which were reverse-transcribed into cDNA following the manufacturer's protocol (Qiagen). cDNA was diluted and distributed to parallel wells for quantitative PCR using gene-specific primers for SLAMF1 or GUSB (Qiagen) and SYBR Green reagent (Qiagen). qPCR was performed following the manufacture's recommended protocol for a total of 40 cycles of 95° C. 15 seconds, 55° C. for 30 seconds, and 72° C. for 30 seconds. Expression within each tissue was normalized to the relative expression of GUSB using the ΔΔCt method. SLAMF1 mRNA was found to be expressed in the thymus, and on CD4+T cells (both naïve and memory, with higher expression on memory T cells), CD8+cytotoxic T cells, T regulatory cells, B cells, and monocyte-derived dendritic cells. See FIG. 3. This RT-PCR data is in agreement with published protein expression data. See, e.g., Aversa et al., 1997, J Immunol 158:4036-4044; Punnonen et al., 1997, J Exp Med 185: 993-1004; Bleharski et al., 2001, J Immunol 167: 3174-3181. These data show that expression of SLAMF1 is enriched on activated T cells, B cells and dendritic cells—a pattern consistent with other immune checkpoints, including PD-1 and TIM3.

### Example 5:

### SLAMF1 Increases Tumor Growth In Vivo

Eleven week old female C57BL/6 mice were purchased from Charles River Laboratories (Hollister, Calif.) and were acclimated for nine days before the start of the study. Mice were treated to induce constitutive, systemic expression of SLAMF1 ECD Fc, using tail vein transfection of a nucleic acid encoding SLAMF1 ECD Fc. Control animals were treated with saline to mimic the induction of gene expression. Next, the murine T-cell lymphoma cell line E.G7-OVA was implanted subcutaneously over the right flank of the mice at 1×10⁶ cells/100 μl/mouse. The E.G7-OVA cell line was purchased from ATCC (Manassas, Va.; Cat. No. CRL-2113). Prior to inoculation, the cells were cultured for three passages in RPMI 1640 medium supplemented with 10% heat-inactivated Fetal Bovine Serum (FBS), 2mM L-Glutamine, 1.5 g/L sodium bicarbonate, 4.5 g/L glucose, 10 mM HEPES, 1.0 mM sodium pyruvate, 0.05 mM 2-mercaptoethanol, 0.4 mg/ml G418, and Antibiotic-Antimycotic solution. Cells were grown at 37° C. in a humidified atmosphere with 5% CO₂. Upon reaching 80-85% confluence, cells were harvested and resuspended in cold Ca²⁺ and Mg²⁺ free phosphate buffered saline (PBS) containing 50% Matrigel at 1×10² cells per milliliter.

Mice were monitored twice weekly following cell implantation for tumor growth. Beginning on Day 5, the length and width of each tumor was measured using calipers and volume was calculated according to the formula: Tumor volume (mm³)=(width (mm)×length (mm))²/2. Tumors continued to be measured at least twice per week until tumor volume exceeded 10% of animal weight, or approximately 2000 mm³. Plasma was collected from all animals at the time of their removal from the study, and expression of SLAMF1 ECD Fc was confirmed by ELISA.

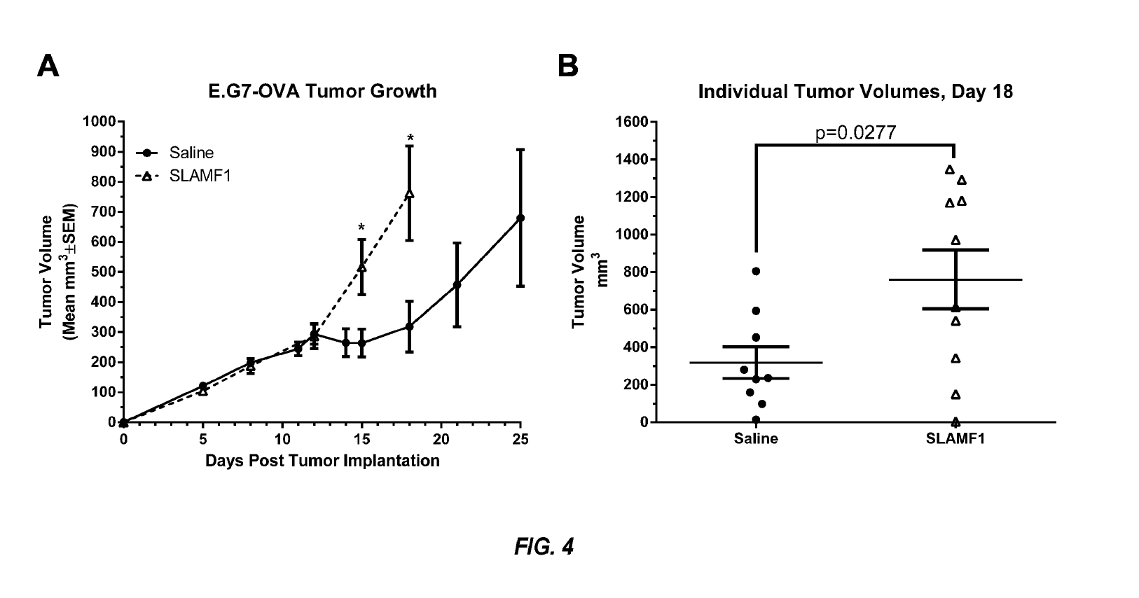

The results of that experiment are shown in FIG. 4. The change in tumor size is shown by graphing mean tumor volume relative to the day upon which animals were inoculated with E.G7-OVA cells. Comparisons of tumor volume as a consequence of treatment with SLAMF1 ECD Fc or saline as a control were determined to be statistically significant if P <0.05. P-values were calculated using unpaired, two-tailed t-test analyses of the calculated tumor volumes on each day upon which tumors were measured. Increased tumor volume in mice that expressed SLAMF1 ECD Fc compared to saline control was statistically significantly on Days 15 (p=0.0305) and 18 (p=0.0277). See FIGS. 4A and 4B.

### Example 6:

### Anti-SLAMF1 Antibodies Relieve Inhibition of T Cells in an Artificial APC Assay

Mouse A20 cells (ATCC TIB-208) were infected with lentivirus containing DNA encoding full-length mouse SLAMF1 (Genecopoeia EX-Mm06571-Lv105) or an empty vector control. Stable expression of mouse SLAMF1 on the A20 cells was confirmed by flow cytometry (BioLegend clone TC15-12F12.2).

The day of the assay, mouse CD4+T cells were isolated from the lymph nodes of D011 mice (Jackson stock number 003303) using a mouse CD4+T cell MACS isolation kit (Miltenyi 130-104-454). The T cells were labeled with CFSE dye (Life Technologies C34554), then washed and counted. A20 cells expressing mouse SLAMF1 or vector control cells were treated with Mitomycin C (Sigma-Aldrich) at 100μg/mL for 1 hour at 37° C. and then washed and counted.

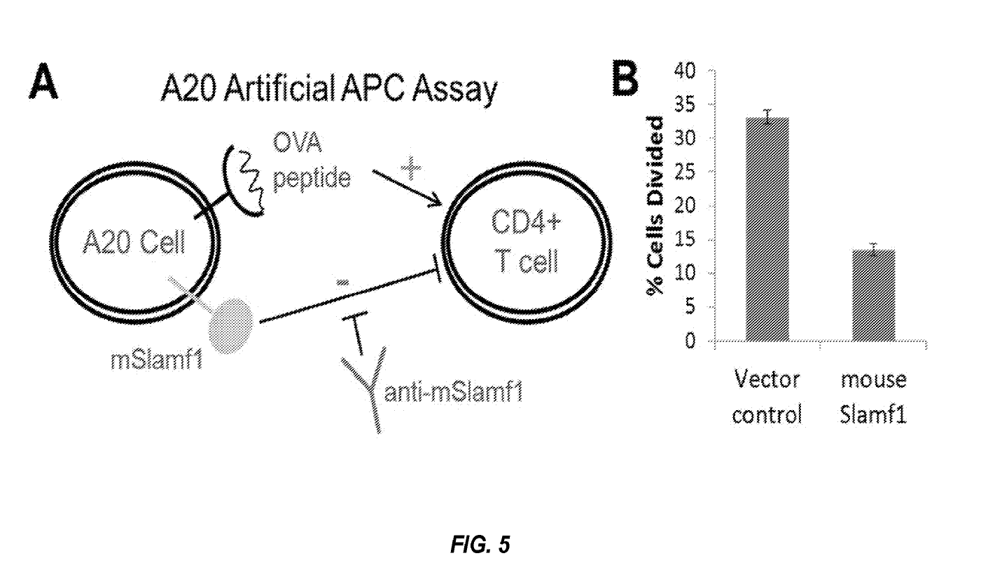

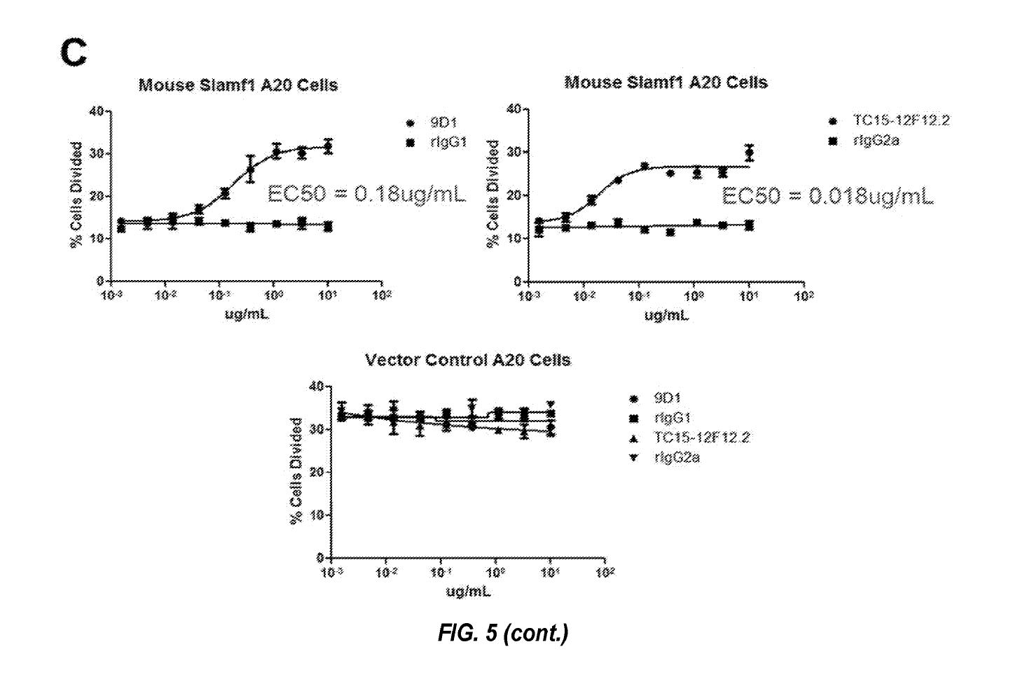

For the assay setup, 100,000 CFSE-labeled CD4+T cells were mixed with 20,000 Mitomycin-C-treated A20 cells and 5ng/mL OVA 323-339 peptide (Anaspec 27024) in RPMI 1640 medium supplemented with 10% heat-inactivated fetal bovine serum (FBS), 2mM L-Glutamine, lx penicillin/streptomycin, lx non-essential amino acids and 0.05mM 2-mercaptoethanol. In some wells, rat anti-mouse SLAMF1 antibodies were added at various concentrations: clone TC15-12F12.2 (BioLegend), clone 9D1 (Affymetrix), or the relevant isotype controls (FIG. 5A). The assay was set up in a 96-well round-bottom plate and incubated at 37° C. and 5% CO2 for 3 days and then CFSE fluorescence was read by flow cytometry. T cell proliferation was analyzed by FlowJo as the percent of T cells that had reduced CFSE fluorescence relative to control wells without OVA peptide (% cells divided).

The results of the experiment are shown in FIG. 5B-C. Transduction of mouse SLAMF1 into the A20 cells reduces the amount of T cell proliferation stimulated by 5ng/mL OVA peptide relative to vector control A20 cells (FIG. 5B). The inhibition is reversed by blocking anti-SLAMF1 monoclonal antibodies (Van Driel et al., 2012 Gastroenterology 143: 1544-1554), while the isotype control antibodies have no effect (FIG. 5C). Further, the anti-SLAMF1 antibodies have no effect when added to T cells mixed with the vector control A20 cells. The results demonstrate that anti-SLAMF1 blocking antibodies reverse co-inhibition of T cell activation by SLAMF1.

### Example 7:

### T Cell Inhibition by Human SLAMF1 in an Artificial APC Assay can be Relieved by Blocking Antibodies

CMV-reactive CD8+T cells were expanded out of HLA-A*02-positive peripheral blood mononuclear cells (PBMCs) from Cellular Technology Ltd using protocols adapted from Gerdemann et al., 2012 Molecular Therapy 20: 1622-32. Briefly, PBMCs were loaded with 10μg/mL CMV pp65 495-503 (Anaspec 28328) for 2 hours at 37° C. and then plated in CTL medium (RPMI 1640 medium supplemented with 10% heat-inactivated human AB serum (Sigma-Aldrich), 2mM L-Glutamine, lx penicillin/streptomycin and 0.05mM 2-mercaptoethanol) supplemented with 2ng/mL recombinant IL-2 (Sigma-Aldrich) and lOng/mL recombinant IL-7 (R&D Systems). After 11 days, the cells were re-stimulated with autologous, Mitomycin-C-treated PHA blasts that were loaded with 10μg/mL CMV peptide (as in Example 6). The cells were allowed to expand for another 10 days and then the CD8+T cells were isolated with the Miltenyi human CD8+T cell isolation kit (130-096-495) and cryopreserved.

Human T2 cells (ATCC CRL-1992) were found to natively express human SLAMF1 by flow cytometry (BioLegend #306308). The day before the assay, a vial of CMV-reactive CD8+T cells was thawed and plated in CTL medium. The day of the assay, T2 cells were loaded with lᵢ.tg/mL CMV peptide in CTL medium for 1 hour at 37° C. with rotation. The cells were then washed 3 times with CTL medium and counted. 100,000 CMV-loaded T2 cells were mixed with 100,000 CMV-reactive CD8+T cells in CTL medium in a 96-well round-bottom plate. Multiple anti-human SLAMF1 antibodies were added into the reaction: a sheep polyclonal (R&D Systems #AF164), mouse clone Al2 (BioLegend #306310), mouse clone IPO-3 (Thermo #MA17626), mouse clone 542301 (R&D Systems #MAB1642), or the appropriate isotype controls for each. The plates were incubated at 37° C. and 5% CO2 for 48 hours and then supernatant was collected and interferon-gamma (IFNγ) levels were measured by Human IFNγ HTRF assay (Cisbio # 62IFNPEB).

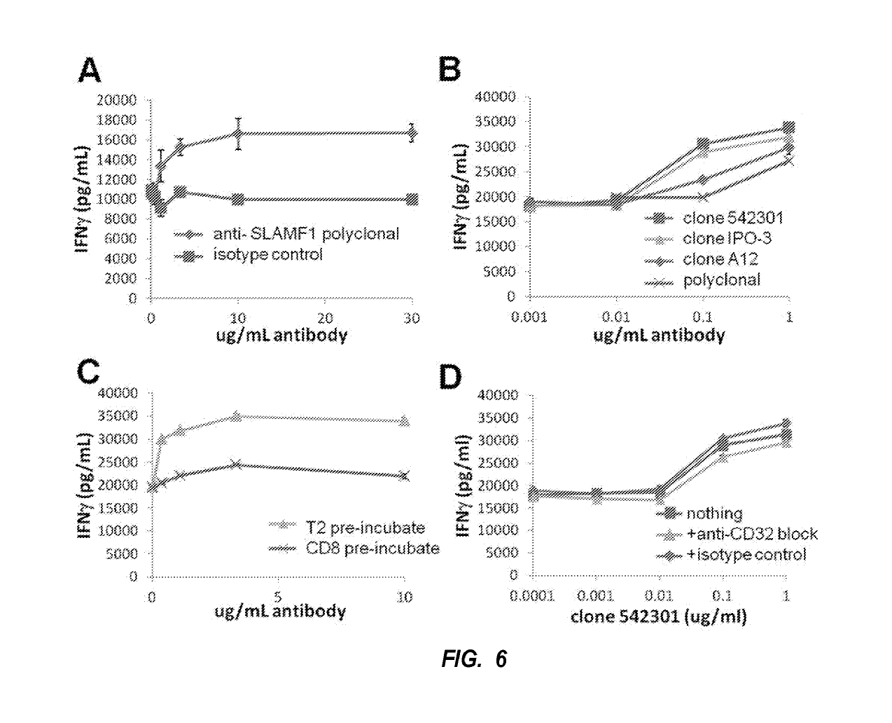

The results of these experiments are shown in FIG. 6. Anti-SLAMF1 polyclonal (FIG. 6A) and monoclonal antibodies (FIG. 6B) stimulate a dose-dependent increase in IFNγ production by the CD8+T cells. To determine which cell type the antibodies worked through, the polyclonal antibody was pre-incubated with either the CMV-loaded T2 cells or with the CD8+T cells and then washed away before setting up the assay. It was found that pre-incubation of the antibody with the T2 cells reproduced the activity of antibody inclusion throughout the assay, whereas pre-incubation of the antibody with the CD8+T cells had minimal effect (FIG. 6C). One possible explanation for why anti-SLAMF1 antibodies are enhancing CD8+T cell activation by T2 cells is that the Fc group of the antibody is cross-linking the CD8+and T2 cells through Fc receptors and increasing activation through this induced proximity. To rule this out, T2 and CD8+T cells were stained by flow cytometry for the presence of Fc receptors. It was found that the T2 cells express one Fc receptor, CD32b. To rule out an effect of the CD32b-mediated cross-linking on the assay, a titration of anti-SLAMF1 clone 542301 was set up with addition of 5μg/mL blocking anti-CD32 antibody (eBioscience #16-0329-85) or isotype control, compared with addition of nothing, at each dose of the anti-SLAMF1 antibody. It was found that addition of neither the CD32 blocking antibody nor the isotype control antibody had any effect on anti-SLAMF1 stimulation of IFN′ release (FIG. 6D).

Taken together, these data indicate that the anti-SLAMF1 antibodies are binding to SLAMF1 and relieving its inhibition of CMV peptide-stimulated activation of CD8+T cells. This activity appears to be primarily due to blocking of SLAMF1 on the T2 cells, not the CD8+cells. This activity is also not due to cross-linking mediated by Fc receptors. These results are consistent with the T cell co-inhibitory activity of SLAMF1 observed in the mouse assays herein.

### Example 8:

### SLAMF1 Is Expressed on Tumor-Infiltrating T Cells

BALB/c mice were purchased from Charles River Laboratories and after acclimation were inoculated with the murine colon carcinoma cell line CT26 (ATCC CRL-2638) subcutaneously over the right flank at lx10⁶ cells/200μ1/mouse. Prior to inoculation, the cells were cultured for no more than three passages in RPMI 1640 medium supplemented with 10% Fetal Bovine Serum (FBS). Cells were grown at 37° C. in a humidified atmosphere with 5% CO2. Upon reaching 80-85% confluence, cells were harvested and resuspended in cold RPMI 1640 containing 50% Matrigel at 5×10⁶ cells per milliliter.

Approximately 1-3 weeks after implantation, tumor tissue was harvested and weighed. The tumor was minced with a razor blade and digested with 5mL of 200U/mL of Collagenase, type I (Worthington Bio, cat# LS004196), in RPMI 1640 medium with shaking for 30 minutes at 37° C. The cell suspensions were passed through a 40₁.tm filter, washed and counted. 10⁶ cells from each tumor were treated with 10₁.tg DNAse I (StemCell technologies, cat# 07900) for 15 minutes at room temperature. 50₁. 1,L of the antibody cocktail in Table 3 was added to the cells and incubated for 30 minutes at 4° C. After staining, the cells were washed twice with cold PBS and then 100μ,L of 1:1000 Aqua Live/Dead dye (Life Technologies L34957) was added and incubated for 15-20 minutes at 4° C. After that the cells were washed 3 more times with cold PBS supplemented with 0.5% BSA and 2mM EDTA and then analyzed by flow cytometry.

TABLE 3
Mouse tumor lymphocyte antibody staining panel

| | Antibody | | |
|---|---|---|---|
| Marker - Color | Clone | Source | Cat # |
| CD25 - BV605 | PC61 | BD Biosciences | 563061 |
| NKp46 - PE | 29A1.4 | eBioscience | 12-3351-82 |
| CD8a - PE-AF610 | 5H10 | Life Technologies | MCD0822 |
| CD3e - PerCP-Cy5.5 | 145-2C11 | eBioscience | 45-0031-82 |
| CD19 - AF700 | 1D3 | eBioscience | 56-0193-82 |
| EphA2 - APC | 233720 | R&D Systems | FAB639A |
| CD11b - APC-Cy7 | M1/70 | BD Biosciences | 557657 |
| CD45 - FITC | 30-F11 | BD Biosciences | 553080 |
| CD4 - BV711 | GK1.5 | BD Biosciences | 563050 |
| SLAMF1 - BV421 | TC15-12F12.2 | BioLegend | 115926 |
| CTLA-4 - PE-Cy7 | UC10-4B9 | BioLegend | 106314 |
| PD1 - BV785 | 29F.1A12 | BioLegend | 135225 |
| Fc block - purified | 2.4G2 | BD Biosciences | 553142 |

For staining regulatory T cells (Tregs) specifically, the tumor cell suspension was stained as above, but with a subset of the antibodies in Table 3: EphA2-APC, CD45-FITC, CD3e-PerCP-Cy5.5, CD4-BV711, CD25-BV605, SLAMF1-BV421 and Fc block. The cells were then washed and fixed with BioLegend FOXP3 Fix/Perm Buffers (catalog #421403) and then stained with anti-mouse FoxP3-PE clone FJK-16s (eBioscience 12-5773-82) or the appropriate isotype control for 30 minutes at room temperature. After that the cells were washed 3 more times with cold PBS supplemented with 0.5% BSA and 2mM EDTA and then analyzed by flow cytometry.

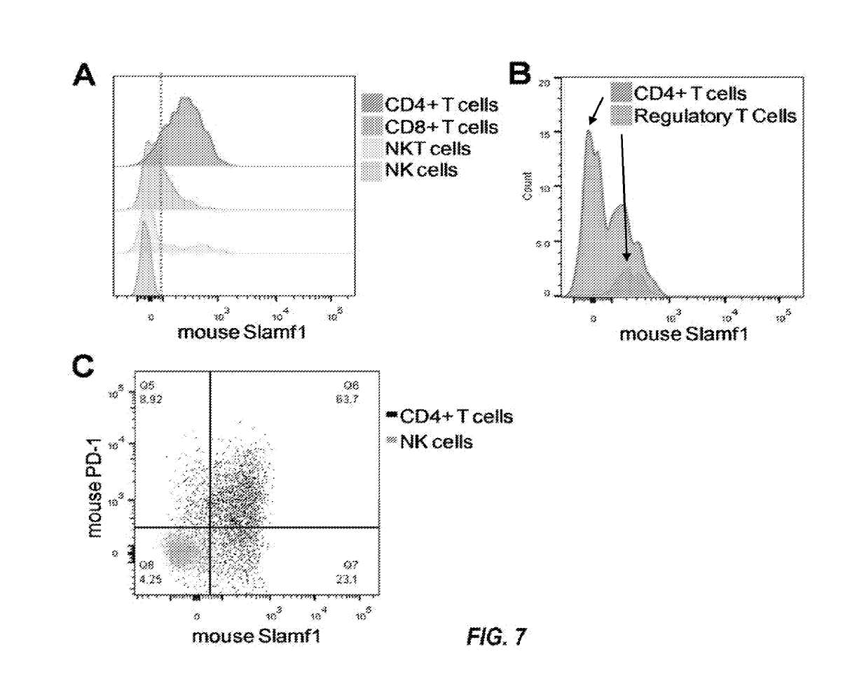

Analysis of the flow cytometry data was carried out in FlowJo with an initial gating for single live cells. The EphA2 and CD45 antibodies were used to identify CT26 tumor cells and the tumor-infiltrating leukocytes, respectively. CD1 lb was used to separate the myeloid cells among the tumor-infiltrating leukocytes, and the remaining antibodies were used to distinguish the various lymphoid cell types. SLAMF1 was found to be expressed on the majority of tumor-infiltrating CD4+T cells, as well as subsets of the CD8+T cells and NKT cells (FIG. 7A). No SLAMF1 expression was observed in NK cells, which are included in FIG. 7 as a negative staining control. Nearly all of the Treg cells were found to express SLAMF1 (FIG. 7B). In addition, the majority of CD4 T cells in the tumor were found to co-express SLAMF1 and PD-1, confirming that these two markers indeed have largely overlapping expression profiles (FIG. 7C, NK cells are included as a negative staining control). Taken together, these data indicate that SLAMF1 is expressed on the majority of CD4+T cells in CT26 tumors, including on the activated T cells and Tregs.

Similar results were obtained when staining MC38 murine colon carcinoma tumors grown in C57BL/6 mice (Charles River).

TABLE OF SEQUENCES

| SEQ | | |
|---|---|---|
| ID NO | Description | Sequence |
| 1 | Human SLAMF1 | MDPKGLLSLT FVLFLSLAFG ASYGTGGRMM |
| | (precursor, with signal | NCPKILRQLG SKVLLPLTYE RINKSMNKSI |
| | peptide, amino acids, 1-20) | HIVVTMAKSL ENSVENKIVS LDPSEAGPPR |
| | (UniProtKB Ref. | YLGDRYKFYL ENLTLGIRES RKEDEGWYLM |
| | Q13291.1, 1 Oct. 2014) | TLEKNVSVQR FCLQLRLYEQ VSTPEIKVLN |
| | | KTQENGTCTL ILGCTVEKGD HVAYSWSEKA |
| | | GTHPLNPANS SHLLSLTLGP QHADNIYICT |
| | | VSNPISNNSQ TFSPWPGCRT DPSETKPWAV |
| | | YAGLLGGVIM ILIMVVILQL RRRGKTNHYQ |
| | | TTVEKKSLTI YAQVQKPGPL QKKLDSFPAQ |
| | | DPCTTIYVAA TEPVPESVQE TNSITVYASV |
| | | TLPES |
| 2 | Human SLAMF1 | ASYGTGGRMM NCPKILRQLG SKVLLPLTYE |
| | (mature, without signal peptide) | RINKSMNKSI HIVVTMAKSL ENSVENKIVS |
| | | LDPSEAGPPR YLGDRYKFYL ENLTLGIRES |
| | | RKEDEGWYLM TLEKNVSVQR FCLQLRLYEQ |
| | | VSTPEIKVLN KTQENGTCTL ILGCTVEKGD |
| | | HVAYSWSEKA GTHPLNPANS SHLLSLTLGP |
| | | QHADNIYICT VSNPISNNSQ TFSPWPGCRT |
| | | DPSETKPWAV YAGLLGGVIM ILIMVVILQL |
| | | RRRGKTNHYQ TTVEKKSLTI YAQVQKPGPL |
| | | QKKLDSFPAQ DPCTTIYVAA TEPVPESVQE |
| | | TNSITVYASV TLPES |
| 3 | Human SLAMF1 | MDPKGLLSLT FVLFLSLAFG ASYGTGGRMM |
| | extracellular domain (ECD) | NCPKILRQLG SKVLLPLTYE RINKSMNKSI |
| | (with signal peptide) | HIVVTMAKSL ENSVENKIVS LDPSEAGPPR |
| | | YLGDRYKFYL ENLTLGIRES RKEDEGWYLM |
| | | TLEKNVSVQR FCLQLRLYEQ VSTPEIKVLN |
| | | KTQENGTCTL ILGCTVEKGD HVAYSWSEKA |
| | | GTHPLNPANS SHLLSLTLGP QHADNIYICT |
| | | VSNPISNNSQ TFSPWPGCRT DPSETKP |
| 4 | Human SLAMF1 | ASYGTGGRMM NCPKILRQLG SKVLLPLTYE |
| | extracellular domain (ECD) | RINKSMNKSI HIVVTMAKSL ENSVENKIVS |
| | (without signal peptide) | LDPSEAGPPR YLGDRYKFYL ENLTLGIRES |
| | | RKEDEGWYLM TLEKNVSVQR FCLQLRLYEQ |
| | | VSTPEIKVLN KTQENGTCTL ILGCTVEKGD |
| | | HVAYSWSEKA GTHPLNPANS SHLLSLTLGP |
| | | QHADNIYICT VSNPISNNSQ TFSPWPGCRT |
| | | DPSETKP |
| 5 | Human SLAMF1 | ASYGTGGRMM NCPKILRQLG SKVLLPLTYE |
| | ECD-Fc, without signal peptide | RINKSMNKSI HIVVTMAKSL ENSVENKIVS |
| | | LDPSEAGPPR YLGDRYKFYL ENLTLGIRES |
| | | RKEDEGWYLM TLEKNVSVQR FCLQLRLYEQ |
| | | VSTPEIKVLN KTQENGTCTL ILGCTVEKGD |
| | | HVAYSWSEKA GTHPLNPANS SHLLSLTLGP |
| | | QHADNIYICT VSNPISNNSQ TFSPWPGCRT |
| | | DPSETKPGSE PKSSDKTHTC PPCPAPELLG |
| | | GPSVFLFPPK PKDTLMISRT PEVTCVVVDV |
| | | SHEDPEVKFN WYVDGVEVHN AKTKPREEQY |
| | | NSTYRVVSVL TVLHQDWLNG KEYKCKVSNK |
| | | ALPAPIEKTI SKAKGQPREP QVYTLPPSRD |
| | | ELTKNQVSLT CLVKGFYPSD IAVEWESNGQ |
| | | PENNYKTTPP VLDSDGSFFL YSKLTVDKSR |
| | | WQQGNVFSCS VMHEALHNHY TQKSLSLSPG |
| | | K |
| 6 | Fc C237S | EPKSSDKTHT CPPCPAPELL GGPSVFLFPP |
| | | KPKDTLMISR TPEVTCVVVD VSHEDPEVKF |
| | | NWYVDGVEVH NAKTKPREEQ YNSTYRVVSV |
| | | LTVLHQDWLN GKEYKCKVSN KALPAPIEKT |
| | | ISKAKGQPRE PQVYTLPPSR DELTKNQVSL |
| | | TCLVKGFYPS DIAVEWESNG QPENNYKTTP |
| | | PVLDSDGSFF LYSKLTVDKS RWQQGNVFSC |
| | | SVMHEALHNH YTQKSLSLSP GK |
| 7 | Exemplary Fc #1 | ERKCCVECPP CPAPPVAGPS VFLFPPKPKD |
| | | TLMISRTPEV TCVVVDVSHE DPEVQFNWYV |
| | | DGVEVHNAKT KPREEQFNST FRVVSVLTVV |
| | | HQDWLNGKEY KCKVSNKGLP APIEKTISKT |
| | | KGQPREPQVY TLPPSREEMT KNQVSLTCLV |
| | | KGFYPSDIAV EWESNGQPEN NYKTTPPMLD |
| | | SDGSFFLYSK LTVDKSRWQQ GNVFSCSVMH |
| | | EALHNHYTQK SLSLSPGK |
| 8 | Exemplary Fc #2 | ESKYGPPCPS CPAPEFLGGP SVFLFPPKPK |
| | | DTLMISRTPE VTCVVVDVSQ EDPEVQFNWY |
| | | VDGVEVHNAK TKPREEQFNS TYRVVSVLTV |
| | | LHQDWLNGKE YKCKVSNKGL PSSIEKTISK |
| | | AKGQPREPQV YTLPPSQEEM TKNQVSLTCL |
| | | VKGFYPSDIA VEWESNGQPE NNYKTTPPVL |
| | | DSDGSFFLYS RLTVDKSRWQ EGNVFSCSVM |
| | | HEALHNHYTQ KSLSLSLGK |

1. A method of treating cancer comprising administering to a subject with cancer an effective amount of at least one SLAMF1 antagonist.

2. A method of inhibiting suppression of activated T cells in a subject comprising administering to the subject at least one SLAMF1 antagonist.

3. The method of claim 1 or claim 2, wherein the method further comprises administering to the subject an effective amount of a therapeutic agent selected from chemotherapeutic agents, anti-angiogenesis agents, growth inhibitory agents, and anti-neoplastic compositions.

4. The method of claim 3, wherein the anti-neoplastic composition comprises an immune stimulating agent.

5. The method of claim 4, wherein the immune stimulating agent is chosen from agents falling within one or more of the following categories:
a) an agonist of an immune stimulatory molecule, including a co-stimulatory molecule, such as an immune-stimulatory molecule found on a T cell or NK cell;
b) an antagonist of an immune inhibitory molecule, including a co-inhibitory molecule, such as an immune-stimulatory molecule found on a T cell or NK cell;
c) an antagonist of CTLA4, LAG-3, PD-1, PDL1, PDL2, Galectin 1, Galectin 9, CEACAM-1, BTLA, CD25, CD69, TIGIT, CD113, GPR56, VISTA, B7-H3, B7-H4, 2B4, CD48, GARP, PD1H, LAIR1, TIM1, TIM3, TIM4, ILT4, IL-6, IL-10, TGFβ, VEGF, KIR, adenosine A2A receptor, PI3Kdelta, or IDO;
d) an agonist of B7-1, B7-2, CD28, 4-1BB (CD137), 4-1BBL, ICOS, ICOS-L, OX40, OX4OL, GITR, GITRL, CD27, CD40, CD4OL, DR3, CD28H, IL-2, IL-7, IL-12, IL-15, IL-21, IFNα, STING, or a Toll-like receptor agonist such as a TLR2/4 agonist;
e) an agent that binds to a member of the B7 family of membrane-bound proteins such as B7-1, B7-2, B7-H2 (ICOS-L), B7-H3, B7-H4, B7-H5 (VISTA), and B7-H6;
f) an agent that binds to a member of the TNF receptor family or a co-stimulatory or co-inhibitory molecule binding to a member of the TNF receptor family such as CD40, CD4OL, OX40, OX4OL, GITR, GITRL, CD70, CD27L, CD30, CD3OL, 4-1BBL, CD137 (4-1BB), TRAIL/Apo2-L, TRAILR1/DR4, TRAILR2/DR5, TRAILR3, TRAILR4, OPG, RANK, RANKL, TWEAKR/Fn14, TWEAK, BAFFR, EDAR, XEDAR, EDA1, EDA2, TACI, APRIL, BCMA, LTβR, LIGHT, DeR3, HVEM, VEGL/TL1A, TRAMP/DR3, TNFR1, TNFβ, TNFR2, TNFα, 1β2, FAS, FASL, RELT, DR6, TROY, or NGFβ;
g) an agent that antagonizes or inhibits a cytokine that inhibits T cell activation such as IL-6, IL-10, TGFβ, VEGF;
h) an agonist of a cytokine that stimulates T cell activation such as IL-2, IL-7, IL-12, IL-15, IL-21, and IFNα; and
i) an antagonist of a chemokine, such as CXCR2, CXCR4, CCR2, or CCR4.

6. A method of inhibiting suppression of activated T cells comprising contacting the T cells with at least one SLAMF1 antagonist.

7. The method of claim 6, wherein the T cells are in vitro.

8. The method of any one of the preceding claims, wherein the at least one SLAMF1 antagonist reduces suppression of proliferation of activated T cells by at least 30%, at least 40%, at least 50%, at least 60%, at least 70%, or at least 80%.

9. The method of claim 8, wherein the activated T cells are CD3+T cells.

10. The method of claim 9, wherein the activated T cells are IL-2-activated CD3+T cells.

11. The method of any one of the preceding claims, wherein the SLAMF1 antagonist is a SLAMF1 extracellular domain (ECD) or a SLAMF1 ECD fusion molecule.

12. The method of claim 11, wherein the SLAMF1 ECD or SLAMF1 ECD fusion molecule is monomeric.

13. The method of claim 11, wherein the SLAMF1 ECD or SLAMF1 ECD fusion molecule is dimeric.

14. The method of any one of claims 1 to 10, wherein the SLAMF1 antagonist is a SLAMF1 antibody.

15. The method of claim 14, wherein the antibody is selected from a chimeric antibody, a humanized antibody, and a human antibody.

16. The method of claim 14, wherein the antibody is a bispecific antibody or a single chain antibody.

17. The method of any one of claims 14 to 16, wherein the antibody is an antibody fragment.

18. The method of claim 17, wherein the antibody fragment is selected from an Fv, a single-chain Fv (scFv), a Fab, a Fab′, and a (Fab′)₂.

19. The method of any one of claims 1 to 10, wherein the SLAMF1 antagonist is a small molecule or a small peptide.

20. A method of identifying a SLAMF1 antagonist, comprising:
c) contacting activated T cells with a candidate molecule and a SLAMF1 molecule, wherein the SLAMF1 molecule comprises SLAMF1, a SLAMF1 ECD, or a SLAMF1 ECD fusion molecule; and
d) detecting proliferation of the activated T cells;
wherein a reduction in suppression of proliferation of the activated T cells in the presence of the candidate molecule as compared to suppression of proliferation of the activated T cells in the absence of the candidate molecule indicates that the candidate molecule is a SLAMF1 antagonist.

21. The method of claim 20, wherein suppression of proliferation of activated T cells is reduced by at least 30%, at least 40%, at least 50%, at least 60%, at least 70%, or at least 80% in the presence of the candidate molecule.

22. The method of claim 20 or claim 21, wherein the candidate molecule binds to SLAMF1.

23. The method of claim 22, wherein the candidate molecule is an antibody that binds SLAMF1.

24. The method of any one of claims 20 to 22, wherein the candidate molecule is a small molecule.

25. The method of any one of claims 20 to 22, wherein the candidate molecule is a small peptide.

26. The method of any one of claims 20 to 25, wherein the activated T cells are activated CD3+T cells.

27. The method of claim 26, wherein the activated T cells are IL-2-activated CD3 + T cells.

28. A method of determining whether a SLAMF1 antibody is a SLAMF1 antagonist, comprising:
c) contacting activated T cells with the SLAMF1 antibody and a SLAMF1 molecule, wherein the SLAMF1 molecule comprises SLAMF1, a SLAMF1 ECD, or a SLAMF1 ECD fusion molecule; and
d) detecting proliferation of the activated T cells;
wherein a reduction in suppression of proliferation of the activated T cells in the presence of the SLAMF1 antibody as compared to suppression of proliferation of the activated T cells in the absence of the SLAMF1 antibody indicates that the SLAMF1 antibody is a SLAMF1 antagonist.

29. The method of claim 28, wherein suppression of proliferation of activated T cells is reduced by at least 30%, at least 40%, at least 50%, at least 60%, at least 70%, or at least 80% in the presence of the SLAMF1 antibody.

30. The method of claim 28 or claim 29, wherein the activated T cells are activated CD3+T cells.

31. The method of claim 30, wherein the activated T cells are IL-2-activated CD3+ T cells.

32. Use of a SLAMF1 antagonist for treating cancer in a subject.

33. The use of claim 32, wherein the SLAMF1 antagonist is a SLAMF1 antibody.

34. The use of claim 33, wherein the antibody is selected from a chimeric antibody, a humanized antibody, and a human antibody.

35. The use of claim 33 or claim 34, wherein the antibody is an antibody fragment.

36. The use of claim 35, wherein the antibody fragment is selected from an Fv, a single-chain Fv (scFv), a Fab, a Fab′, and a (Fab′)2.

37. The use of claim 33, wherein the antibody is a bispecific antibody or a single chain antibody.

38. The use of claim 32, wherein the SLAMF1 antagonist is a SLAMF1 extracellular domain (ECD) or a SLAMF1 ECD fusion molecule.

39. The use of claim 38, wherein the SLAMF1 ECD or SLAMF1 ECD fusion molecule is monomeric.

40. The use of claim 38, wherein the SLAMF1 ECD or SLAMF1 ECD fusion molecule is dimeric.

41. The use of claim 32, wherein the SLAMF1 antagonist is a small molecule or a small peptide.
